(19) United States
(12) Patent Application Publication
(10) Pub. No.: US 2005/0283147 A1
(43) Pub. Date: Dec. 22, 2005

(54) Surgical treatment device and surgical treatment system

(76) Inventor: Chie Yachi, Tokyo (JP)

Correspondence Address: OSTROLENK FABER GERB & SOFFEN, 1180 AVENUE OF THE AMERICAS, NEW YORK, NY, 100368403, US

(21) Appl. No.: 11/155,047

(22) Filed: Jun. 17, 2005

(30) Foreign Application Priority Data
Jun. 17, 2004 (JP) 2004-180188
Jun. 17, 2004 (JP) 2004-180189

(51) Int. Cl. A61B 18/04

(52) U.S. Cl. 606/28

(57) ABSTRACT

A surgical treatment device includes a shaft, a treatment portion which is provided at the tip of the shaft to effect heat treatment to living tissues, heater elements which are disposed in the treatment portion, a cable member which is inserted through the shaft to supply power to the heater elements, and a heat insulating member for inhibiting heat transfer from the treatment portion to the shaft. As the heater elements are disposed in the treatment portion consisting of a member having high heat conductivity, the heat generated by the heater elements can be efficiently utilized. In addition, as the heat transfer to the shaft is inhibited by a heat insulating member, heat loss is small. According to the surgical treatment device, coagulation treatment and blunt separation treatment can be efficiently carried out.

### CROSS-REFERENCE TO RELATED APPLICATION

This application is related to Japanese Patent Applications No. 2004-180188 filed on Jun. 17, 2004, and No. 2004-180189 filed on Jun. 17, 2004, the contents of which are hereby incorporated by reference.

### BACKGROUND OF THE INVENTION

1. Field of the Invention

The present invention relates to a surgical treatment device. More particularly, this invention relates to a surgical treatment device for carrying out treatments, such as coagulation treatment and separation treatment by cauterizing living tissues in a body cavity, and to a surgical treatment system including such a surgical treatment device.

2. Description of Related Art

Some surgical treatment devices have been widely known, including a device for cauterizing organs in a body cavity, such as respiratory organs and digestive organs, or a mucous membrane or the like in a nose, and a device for cauterizing parenchymal organs, such as a liver, or for performing blunt separation of organs from connective tissues or fat tissues. Various suggestions have been made for such surgical treatment devices.

Japanese Unexamined Published Application No. 11-197158 describes a surgical treatment device (cautery probe) which can be inserted through an endoscope. This surgical treatment device has a cylindrical coagulation treatment portion at its tip. Coagulation treatment can be given by pressing the peripheral surface of the coagulation treatment portion onto the tissues in a body cavity.

A surgical treatment device described in Japanese Unexamined Published Application No. 2002-248112 has a heater element at the tip of an insertion portion, and a heat transfer member which is thermally coupled to the heater element and projected further than the tip of the insertion portion. Tissues in a body cavity can be coagulated or incised by the transfer of heat generated by the heater element to the outer peripheral surface of a treatment portion through the heat transfer member.

A surgical treatment device disclosed in U.S. Publication No. 2002/0062123 is configured such that a high-frequency current is passed through a conductive fluid dropped from a treatment portion to thereby electrically effect heat treatment of tissues.

A cautery probe disclosed in Japanese Unexamined Published Application No. 2001-112772 has a heat insulating configuration wherein heat generated by a heater element is inhibited from being transferred to the outer surface of a cap which requires no heating, and is provided at the tip of the cautery probe.

The surgical treatment device (cautery probe) disclosed in Japanese Unexamined Published Application No. 11-197158 is required to be inserted through an endoscope, which is not in a form necessarily appropriate for surgical operation, e.g. such treatment as blood coagulation or separation of tissues across a wide range.

The surgical treatment device disclosed in this publication provides a soft cautery probe covered with a soft tube. In providing a rigid cautery probe, the cautery probe is required to be covered, for example, with a rigid metal tube instead of the soft tube. This, however, creates a problem of reducing thermal efficiency of the cautery probe, since heat of a heater element is unavoidably transferred to the metal tube.

The surgical treatment device disclosed in Japanese Unexamined Published Application No. 2002-248112 is likely to cause heat loss because heat of a heater element is permitted to be transferred to the outer peripheral surface of the treatment portion through the heat transfer member. Thus, the device is considered to have room for improvement in heat efficiency. In other words, the means disclosed in this publication causes heat loss as well, because heat is radiated from a heater element to portions other than the portion being treated. This creates a problem in that a long time is required for finishing coagulation treatment or separation treatment which is the original role of the device.

The surgical treatment device disclosed in U.S. Publication No. 2002/0062123 requires, when used, to constantly drop a conductive fluid, which increases time and costs for preparation before the device is actually used. Further, allowing the conductive fluid to flow out is likely to entail unpredictable denaturation of tissues.

The cautery probe disclosed in Japanese Unexamined Published Application No. 2001-112772 has a heat insulating portion, which is for inhibiting heating of portions of the outer surface of the cap provided at the tip of the cautery probe other than the outer surface of a cautery portion. The cautery probe is not equipped with means for inhibiting transfer of heat generated by the heater element to portions other than the cap. Thus, the cautery probe causes heat loss due to heat radiation to the portions other than the cap, thus arising a problem that so much the more time is required for coagulation treatment.

### SUMMARY OF THE INVENTION

This invention has been made in view of the circumstances described above, and has as its object to provide a surgical treatment device and a surgical treatment system, which hardly cause heat loss and thus can efficiently effect coagulation treatment and blunt separation treatment.

In order to accomplish the object mentioned above, the surgical treatment device of the present invention includes:
a shaft;
a treatment portion provided at a tip of the shaft for effecting heat treatment to living tissues;
a heater disposed within the treatment portion;
a cable member inserted through the shaft for supplying electricity to the heater: and
a heat insulating member inhibiting heat transfer from the treatment portion to the shaft.

This configuration is advantageous in that it enables efficient utilization of heat generated by the heater because the heater is disposed in the treatment portion which is formed of a member having high heat conductivity, and that it causes hardly any heat loss compared to conventional surgical treatment because heat is inhibited from being transferred to the shaft by the heat insulating member. Thus, according to the surgical treatment device of the present invention having this configuration, efficient performance is ensured in coagulation treatment and blunt separation treatment.

Another surgical treatment device of the present invention for accomplishing the object mentioned above includes:
a shaft having a grip member at base end side thereof;
a treatment portion provided at a tip of the shaft for effecting heat treatment to living tissues, the treatment portion having a through hole opening to a front end thereof;
a heater disposed within the treatment portion;
a cable member inserted through the shaft for supplying electricity to the heater;
a first conduit member connected to the through hole;
a first connector provided at the grip member and connected to the cable member; and
a second connector provided at the grip member and connected to the first conduit member.

This configuration is advantageous in that it enables efficient utilization of heat generated by the heater compared to conventional surgical treatment devices, because the heater is disposed in the treatment portion consisting of a member having high heat conductivity to cause hardly any heat loss. Thus, according to the surgical treatment device of the present invention having this configuration, efficient performance is ensured in coagulation treatment and blunt separation treatment.

In addition to the advantages described above, this configuration is advantageous in that a user can also carry out an operation of liquid supply, suction or air supply without changing devices. Thus, the surgical treatment device according to the present invention having this configuration, realizes a highly functional surgical treatment system which can shorten operation time.

Other configurations and effects of the present invention will be apparent from the description of the embodiments provided hereunder and the appended drawings.

### BRIEF DESCRIPTION OF THE DRAWINGS

In the drawings:

FIG. 1 is a schematic block diagram of a surgical treatment system including a surgical treatment device according to a first embodiment of the present invention;

FIG. 2 is a schematic half cross-sectional side view showing an internal configuration of an essential part of the surgical treatment device shown in FIG. 1;

FIG. 3 is a cross-sectional side view showing an internal configuration of a tip portion of the surgical treatment device shown in FIG. 1;

FIG. 4 is a vertical cross-sectional view taken along a line IV-IV of FIG. 3;

FIG. 5 is a cross-sectional side view showing an internal configuration of a tip portion of a surgical treatment device according to a second embodiment of the present invention;

FIG. 6 is a cross-sectional side view showing an internal configuration of a tip portion of a surgical treatment device according to a third embodiment of the present invention;

FIG. 7 is a cross-sectional side view showing an internal configuration of a tip portion of a surgical treatment device according to a fourth embodiment of the present invention;

FIG. 8 is a cross-sectional side view showing an internal configuration of a tip portion of a surgical treatment device according to a fifth embodiment of the present invention;

FIG. 9 is a vertical cross-sectional view in the vicinity of a tip portion of a surgical treatment device according to a sixth embodiment of the present invention;

FIG. 10 is a vertical cross-sectional view in the vicinity of a tip portion of a surgical treatment device according to a seventh embodiment of the present invention;

FIG. 11 is a cross-sectional side view showing an internal configuration of a tip portion of a surgical treatment device according to an eighth embodiment of the present invention;

FIG. 12 is a schematic half cross-sectional side view showing an internal configuration of an operating part of the surgical treatment device shown in FIG. 11;

FIG. 13 is a cross-sectional side view showing an internal configuration of a tip portion of a surgical treatment device according to a ninth embodiment of the present invention;

FIG. 14 is a cross-sectional side view showing an internal configuration of a tip portion of a surgical treatment device according to a tenth embodiment of the present invention;

FIG. 15 is a schematic block diagram showing a configuration of a surgical treatment system including a surgical treatment device according to an eleventh embodiment of the present invention;

FIG. 16 is a cross-sectional side view showing an internal configuration of a tip portion of the surgical treatment device shown in FIG. 15;

FIG. 17 is a vertical cross-sectional view taken along a III-III line of FIG. 16;

FIG. 18 is a schematic half cross-sectional side view of an internal configuration of the operating part of the surgical treatment device shown in FIG. 15;

FIG. 19 is a schematic block diagram showing a configuration of a surgical treatment system according to a twelfth embodiment of the present invention;

FIG. 20 is a schematic block diagram showing a configuration of a surgical treatment system according to a thirteenth embodiment of the present invention;

FIG. 21 is a schematic block diagram showing a configuration of a surgical treatment system according to a fourteenth embodiment of the present invention;

FIG. 22 is a cross-sectional side view showing an internal configuration of a tip portion of the surgical treatment device shown in FIG. 21;

FIG. 23 is a schematic half cross-sectional side view showing an internal configuration of an operating part of the surgical treatment device shown in FIG. 21;

FIG. 24 is a vertical cross-sectional view showing a vicinity of a tip portion of a surgical treatment device according to a fifteenth embodiment of the present invention;

FIG. 25 is a vertical cross-sectional view showing a vicinity of a tip portion of a surgical treatment device according to a sixteenth embodiment of the present invention;

FIG. 26 is a cross-sectional side view showing an internal configuration of a tip portion of the surgical treatment device shown in FIG. 25:

FIG. 27 is a cross-sectional side view showing an internal configuration of a tip portion of a surgical treatment device according to a seventeenth embodiment of the present invention;

FIG. 28 is a cross-sectional side view showing an internal configuration of a tip portion of a surgical treatment device according to an eighteenth embodiment of the present invention; and

FIG. 29 is a cross-sectional side view showing an internal configuration of a tip portion of a surgical treatment device according to a nineteenth embodiment of the present invention.

### DETAILED DESCRIPTION OF THE PREFERRED EMBODIMENTS

Hereinafter are described various embodiments of the surgical treatment device and the surgical treatment system of the present invention.

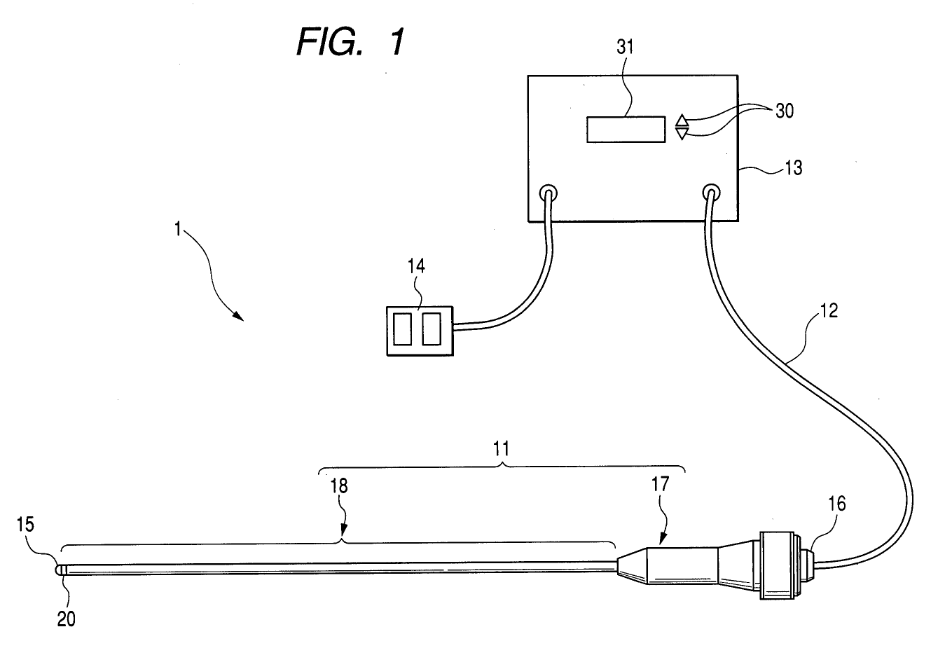

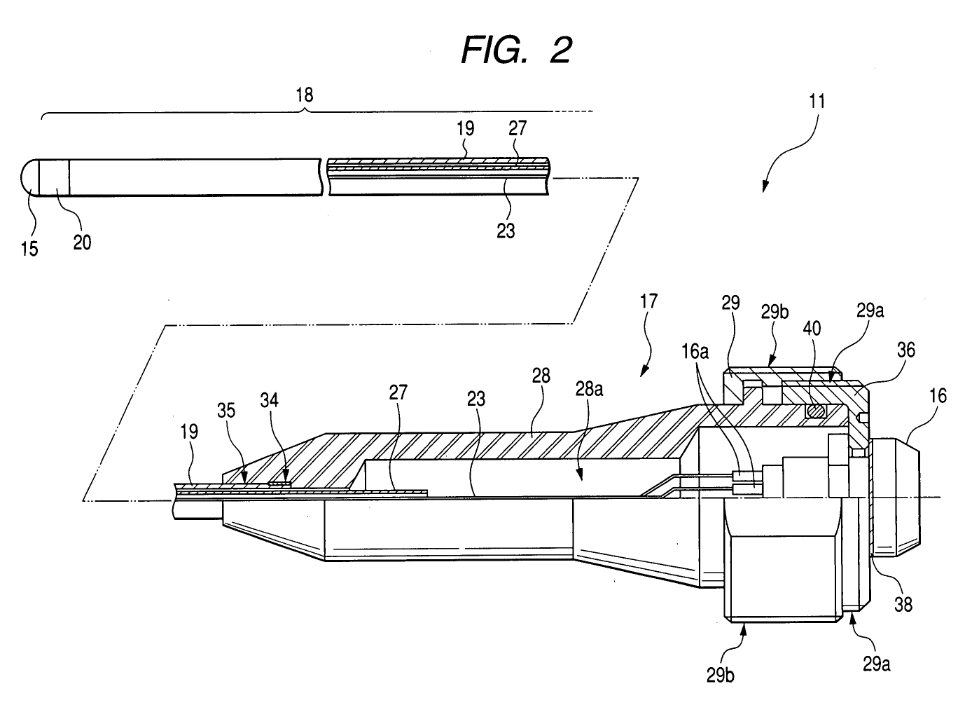

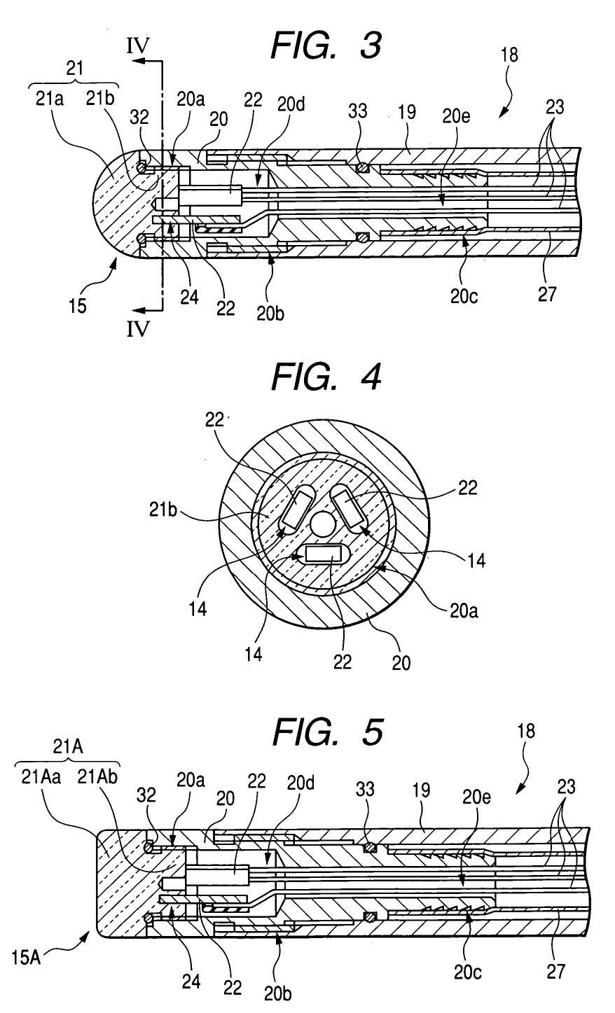

FIG. 1 is a schematic block diagram showing a surgical treatment system including a surgical treatment device according to a first embodiment of the present invention. FIG. 2 is a schematic half cross-sectional side view showing an internal configuration of an essential part of the surgical treatment device of the present embodiment. FIG. 3 is a cross-sectional side view showing an internal configuration of a tip portion of the surgical treatment device of the present invention. FIG. 4 is a vertical cross-sectional view taken along a IV-IV line of FIG. 3.

A surgical treatment device 11 according to the present embodiment is used as a part of a surgical treatment system 1. As shown in FIG. 1, the surgical treatment system 1 mainly includes the surgical treatment device 11 of the present embodiment, a power supply unit 13 which is electrically connected to the surgical treatment device 11 through a connecting cable 12, and a foot switch 14 which is connected to the power supply unit 13 and generates an on-off control signal for supplying power to heater elements 22 by a user's operation, such as a step-on operation.

The surgical treatment device 11 mainly includes an operating part 17 which is a portion gripped by a user and has a connector 16 for connecting the connecting cable 12 thereto, a treatment portion 15 which effects heat treatment to living tissues, and a shaft 18 which links the treatment portion 15 with the operating part 17 and is formed to be insertable into a body cavity.

As shown in FIG. 2, the shaft 18 has rigidity, and includes a tubular member 19 serving as a mantle tube for protecting leads 23 (cable members) which will be described later, and a connecting member 20 which is disposed, for linkage, between the tubular member 19 (mantle tube) and the treatment portion 15. A hollow tube 27 formed of an insulating material is disposed inside the tubular member 19 so as to extend from the rear end the connecting member 20 to the operating part 17. The leads 23 are inserted through the tube 27 (through the shaft 18), extending from the respective heater elements 22 (see FIG. 3, details of which will be described later) in the treatment portion 15. One end of each of these leads 23 is connected to each of the connecting terminals 16a (see FIG. 2) of the connector 16 of the operating part 17.

The power supply unit 13 has a capability of controlling the heat generating operation of the heater elements 22 (see FIG. 3) which are provided inside the treatment portion 15 of the surgical treatment device 11. Specifically, as shown in FIG. 1, on the front panel of the power supply unit 13, a temperature setting button 30 for selectively setting temperature of the treatment portion 15 and a display 31 for displaying a set value or the like set by the temperature setting button 30 are disposed.

By operating the temperature setting button 30, a user can set the temperature of the treatment portion 15 at any level. The power supply unit 13 carries out the control to maintain the temperature of the treatment portion 15 at a set value.

At the distal end of the shaft 18, the treatment portion 15 is provided, as described above, which includes, as shown in FIGS. 3 and 4, an end cap 21 and the heater elements 22 disposed inside the end cap 21, whose resistance varies depending on temperature.

The end cap 21 is made of a material of high heat conductivity, such as copper, molybdenum and platinum. Further, surface treatment, such as Teflon coating®, is given to the outer surface of the end cap 21 so that clinging of tissues may be inhibited when used in a body cavity.

The end cap 21 is formed of a hemispherical portion 21a having a semicircular side section, and a protrusion 21b which is integral with the hemispherical portion 21a and protrudes rearward. A plurality of grooves 24 having a predetermined depth are formed on one circumference of the protrusion 21b with a certain interval therebetween. Each of the heater elements 22 is fixed to each of these grooves 24 by, for example, soldering or brazing, so that the heater elements 22 are thermally coupled to the end cap 21.

The outer peripheral portion of the protrusion 21b of the end cap 21 is engaged with a first engaging portion 20a of the connecting member 20. The connecting member 20 also has a second engaging portion 20b which is formed halfway on its outer peripheral surface. The second engaging portion 20b is engaged with the inner peripheral surface at an end portion of the tubular member 19, whereby the end cap 21 and the tubular member 19 (i.e. shaft 18) are linked with each other through the connecting member 20. An O-ring 32 formed of a waterproof member is provided at a connecting portion between the end cap 21 and the connecting member 20. Also, an O-ring 33 formed of a waterproof member is provided at a connecting portion between the connecting member 20 and the tubular member 19. Thus, water-tightness is ensured at the respective connecting portions.

The connecting member 20 is an annular member made of a material having low heat conductivity and having electric insulating properties. The connecting member 20 includes, as described above, the first engaging portion 20a which is formed on the inner peripheral surface of its end portion and is engaged with the predetermined portion of the end cap 21 (i.e. the outer peripheral surface of the protrusion 21b), the second engaging portion 20b which is formed halfway on its outer peripheral surface and is engaged with the predetermined portion of the tubular member 19 (i.e. the inner peripheral surface at the tip end portion), and a tube joint 20c which is formed on the outer peripheral surface at the rear end portion, for linkage with the tube 27.

One end of the tube 27, which is inserted through the bore of the tubular member 19 in the shaft 18, is connected to the tube joint 20c at the rear end of the connecting member 20. The tube 27 extends to a predetermined position in the operating part 17.

In the bore of the connecting member 20, a large diameter portion 20d is provided at its end side and a small diameter portion 20e is provided at its rear side. A heater elements 22 are partially positioned in the bore of the large diameter portion 20d. Further, the leads 23 extending from the respective heater elements 22 are inserted through the bore of the small diameter portion 20e which communicates with the bore of the large diameter portion 20d. The leads 23 are further inserted through the bore of the tube 27 and connected to the respective connecting terminals 16a (see FIG. 2) of the connector 16 at the operating part 17.

As shown in FIG. 1, the connecting cable 12 is connected to the connector 16. Thus, electrical connection between the heater elements 22 and the power supply unit 13 is ensured. In other words, electrical connection is ensured between the heater elements 22 and the power supply unit 13 through the leads 23, the connector 16 and the connecting cable 12.

As shown in FIG. 4, the plurality of (three in the present embodiment) heater elements 22 are arranged on one circumference centering on the axis of the protrusion 21b of the end cap 21, with a certain interval therebetween. The number of the heater elements 22 are adjusted depending on the heat capacity of the end cap 21.

The operating part 17, as shown in FIG. 2, mainly includes a grip member 28 which is gripped by a user, a clamp member 29 which is provided at the base end side of the grip member 28, and a connector fixing member 36 which fixes the connector 16 through the engagement with the clamp member 29.

The grip member 28 has in its inside a hollow portion 28a. The tubular member 19 extends to the end of this hollow portion 28a. The connecting portion between the grip member 28 and the tubular member 19 is fixed by a locking member 34 and adhesive filler material 35. A waterproof adhesive may be used as the adhesive filler material 35.

As described above, the tube 27 is inserted through the tubular member 19. This tube 27 extends further rearward from the rear end of the tubular member 19. The rear end of the tube 27 is positioned approximately midway of the grip member 28. In the hollow portion 28a of the grip member 28, the leads 23 which are inserted through the bore of the tube 27 are connected to the respective connecting terminals 16a of the connector 16. Thus, the leads 23 are sufficiently protected from the impact from outside.

The clamp member 29 is provided with the engaging portion 29a at the inner periphery of its gripping portion. The engaging portion 29a is engaged with the connector fixing member 36 for fixing the connector 16. Specifically, the connector fixing member 36 is positioned between the engaging portion 29a of the clamp member 29 and the outer peripheral surface of the rear end portion of the grip member 28. An O-ring 40 formed of a waterproof member is provided between the grip member 28 and the connector fixing member 36. Further, a sealing member 38 having waterproof properties is provided between the connector fixing member 36 and the connector 16.

The engaging portion 29b is formed at the outer periphery of the clamp member 29b which is engaged with a protection cover (not shown) for protecting the connector 16. By attaching this protection cover using the engaging portion 29b of the clamp member 29, the surgical treatment device 11 can be dipped in an antiseptic chemical, for example.

Hereinafter is described the operation of the surgical treatment system including the surgical treatment device 11 of the present embodiment configured as described above.

First of all, a user can connect the surgical treatment device 11 and a foot switch 14 to the power supply unit 13. Thereafter, a start-up preparation can be carried out, including turning on the power supply unit 13.

After having the surgical treatment system ready for use, a user can operate a temperature setting button 30 of the power supply unit 13 to set a temperature of the treatment portion 15 at an optimum level for the treatment to be given. At this moment, the result of setting, namely the set temperature or the set level is indicated on the display 31.

A user can then hold the operating part 17 of the surgical treatment device 11, and can then allow the treatment portion 15 of the surgical treatment device 11 to touch the tissues in a body cavity, followed by stepping on the foot switch 14. Thus, electrical power is supplied to the heater elements 22 from the power supply unit 13 to allow the heater elements 22 to generate heat.

The heater elements 22, as described above, are characteristic in that their resistance is varied depending on temperature. Accordingly, the power unit 13 can control the temperature of the heater elements 22 so as to be kept at a level set by a user, by monitoring the resistance of the heater elements 22.

Heat generated by the heater elements 22 is transferred to the end cap 21, by which the treatment portion 15 is heated. The heater elements 22 are directly coupled to the end cap 21 as described above. Therefore, no heat loss is caused in the transfer of heat from the heater elements 22 to the end cap 21. Thus, the temperature of the treatment portion 15 becomes approximately the same as the set temperature.

The heater elements 22 are disposed on one circumference in the end cap 21 with approximately an even interval therebetween. The number and positions of the heater elements 22 can be changed depending on the heat capacity of the end cap 21. The end cap 21 is formed of a material having high heat conductivity, and the connecting member 20 to be linked to the treatment portion 15 is formed of a material having low conductivity. In this way, as the treatment portion 15 is thermally insulated from outside, the temperature of the outer surface of the treatment portion 15 is approximately uniformed. Thus, if any portion on the outer surface of the treatment portion 15 is allowed to contact with the tissues in a body cavity, the same effects can be given to the tissues.

As is apparent from the description provided above, the connecting member 20, being disposed between the treatment portion 15 having the heater elements 22 and the tubular member 19 (mantle tube), serves as a linkage between the both, and at the same time, serves as heat insulating means for inhibiting heat transfer between the both.

As described above, surface treatment, such as Teflon coating® is given to the outer surface of the end cap 21, and the connecting member 20 is formed of a material having low heat conductivity. For these reasons, the temperature of the connecting member 20 is not so increased as the treatment portion 15. Accordingly, if the tissues of a body cavity are in contact with the outer surfaces of the end cap 21 and the connecting member 20, clinging of tissues onto the outer surfaces is suppressed.

Further, the treatment portion 15, the connecting member 20, the shaft 18 and the operating part 17 are integrally linked, and these are configured into a shape of a rod having rigidity. Thus, when a user holds and moves the operating part 17, the treatment portion 15 moves accordingly. In this way, a user can readily move the treatment portion 15 to a target position by operating the operating part 17.

By stepping on the foot switch 14 during the operation of the operating part 17, a user can efficiently effect blunt separation of the tissues with the treatment portion 15 while effecting coagulation treatment.

As described above, an operator can safely and efficiently perform coagulation treatment and blunt separation treatment, according to the first embodiment.

It should be noted that, in the first embodiment, the tubular member 19 of the shaft 18 is described as having rigidity and a linearly extending shape, however, the tubular member 19 may also have a rigidly curved or bent shape. Alternatively, the tubular member 19 may be made of bendable or flexible material.

In the first embodiment, the end cap 21 of the treatment portion 15 is described as having a hemispherical shape, however, the end cap 21 may have various shapes as exemplified in second to fifth embodiments described below.

The surgical treatment devices of the following second to fifth embodiments have substantially the same configuration as the surgical treatment device 11 in the first embodiment. The only difference between the second to fifth embodiments and the first embodiment is the shape of the end cap. Accordingly, the constructional elements of the second to fifth embodiments, having the similar configurations to those of the constructional elements of the first embodiment, are referred to by the same reference numbers, and descriptions therefor are omitted.

FIG. 5 is a cross-sectional side view showing an internal configuration of a tip portion of a surgical treatment device according to a second embodiment of the present invention.

A treatment portion 15A of the surgical treatment device of the present embodiment includes, as in the first embodiment, an end cap 21A, and the heater elements 22 disposed inside the end cap 21A.

The end cap 21A is formed of a cylindrical portion 21Aa whose end surface is substantially flat, and a protrusion 21Ab which is integral with the cylindrical portion 21Aa and protrudes rearward. As in the first embodiment, the plurality of grooves 24 for fixing the plurality of heater elements 22, respectively, are formed at the protrusion 21Ab.

The configuration of the present embodiment is the same as the first embodiment except as described above. The operation is also the same as the first embodiment.

In the second embodiment having the configuration as described above, safe and efficient coagulation treatment can be given as in the first embodiment. Moreover, in the present embodiment, when giving treatment along a longitudinal axis of the treatment portion 15A, a user can cover tissues across a broader range.

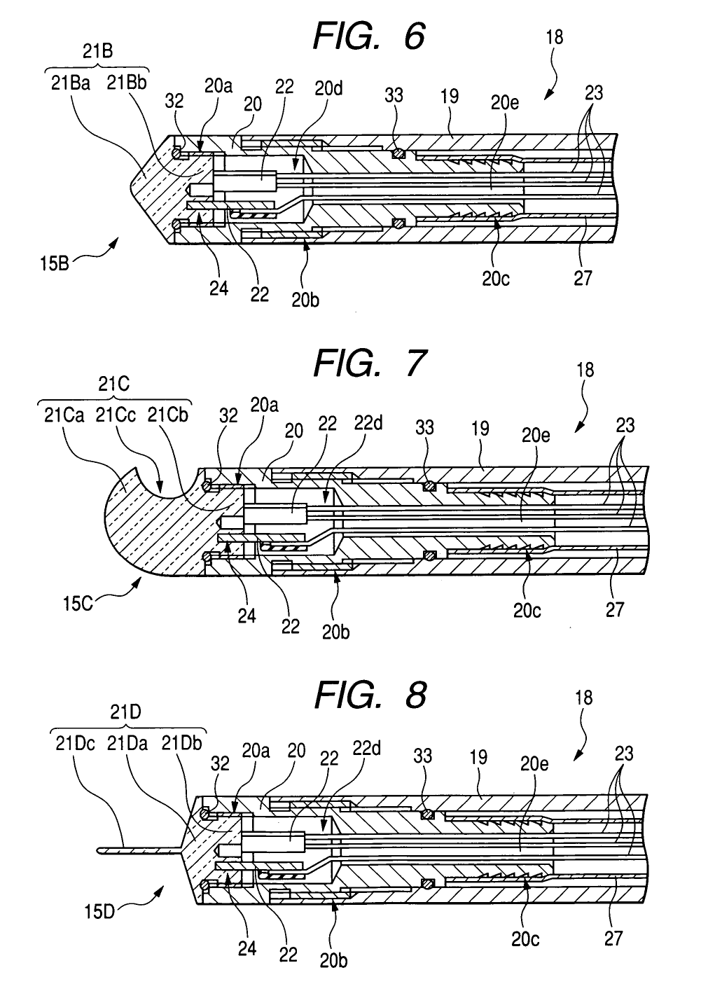

FIG. 6 is a cross-sectional side view showing an inner configuration of a tip portion of a surgical treatment device according to a third embodiment of the present invention.

A treatment portion 15B of the surgical treatment device of the present embodiment includes, as in the first embodiment, an end cap 21B, and the heater elements 22 disposed inside the end cap 21B.

The end cap 21B is formed of a conical portion 21Ba whose side cross section is substantially triangle, and a protrusion 21Bb which protrudes rearward from the conical portion 21Ba. As in the first embodiment, the plurality of grooves 24 for fixing the plurality of heater elements 22 thereto are formed at the protrusion 21Bb.

The configuration of the present embodiment is the same as the first embodiment except as described above. The operation is also the same as the first embodiment.

In the third embodiment having the configuration as described above, safe and efficient coagulation treatment can be effected as in the first embodiment. Moreover, the conically shaped end cap 21B of the present embodiment readily enables delicate separation treatment of living tissues or penetration operation into tissues.

FIG. 7 is a cross-sectional side view showing an inner configuration of a tip portion of a surgical treatment device according to a fourth embodiment of the present invention.

A treatment portion 15C of the surgical treatment device of the present embodiment includes an end cap 21C, and the heater elements 22 disposed inside the end cap 21C.

The end cap 21C is formed of a hooked portion 21Ca which is provided with a notch 21Cc having a semicircular cross section, and a protrusion 21Cb which is integral with the hooked portion 21Ca and protrudes rearward.

As in the first embodiment, the plurality of grooves 24 for fixing the plurality of heater elements 22, respectively, are formed at the protrusion 21Cb.

The configuration of the present embodiment is the same as the first embodiment except as described above. The operation is also the same as the first embodiment.

According to the fourth embodiment having the configuration as described above, safe and efficient coagulation treatment can be effected as in the first embodiment. In the present embodiment, a user can more readily carry out separation operation of tissues by utilizing the notch 21Cc of the end cap 21C.

FIG. 8 is a cross-sectional side view showing an inner configuration of a tip portion of a surgical treatment device according to a fifth embodiment of the present invention.

A treatment portion 15D of the surgical treatment device of the present embodiment includes an end cap 21D, and the heater elements 22 disposed inside the end cap 21D.

The end cap 21D, whose side cross section has a shape of obtuse triangle, is formed of a needle-bearing conical portion 21Da having a needle-shaped portion 21Dc at its distal end, and a protrusion 21Db which is integral with the needle-bearing conical portion 21Da and protrudes rearward. As in the first embodiment, the protrusion 21Db is provided with the plurality of grooves 24 for fixing the plurality of elements 22, respectively.

The configuration of the present embodiment is the same as the first embodiment except as described above.

The operation is also the same as the first embodiment. The surgical treatment device of the present embodiment is used such that electric power is supplied to the heater elements 22 from the power supply unit 13 after stinging the needle-like portion 21Dc into tissues. According to the present embodiment, tissues can be heated and coagulated in their insides.

According to the fifth embodiment having the configuration as described above, safe and efficient coagulation treatment can be effected as in the first embodiment. Moreover, the present embodiment readily enables a user to carry out penetration/cautery treatment whereby, tissues are heated and coagulated locally or only in their insides.

In the second to the fifth embodiments, as in the first embodiment, the end caps 21A, 21B, 21C and 21D are formed of materials of high heat conductivity, and the outer surfaces of the end caps 21A, 21B, 21C and 21D are given with surface treatment which may inhibit clinging of tissues.

In addition, each of the outer peripheral portions of the protrusions 21Ab, 21Bb, 21Cb and 21Db of the end caps 21A, 21B, 21C and 21D, respectively, is engaged with the first engaging portion 20a of the connecting member 20.

Various forms of an end cap can be suggested as described above. Moreover, each of the end caps, per se, may be made of heater elements.

In the first embodiment, the heater elements 22 of the treatment portion 15 are disposed on one circumference with a certain interval therebetween. The arrangement of the heater elements 22, however, is not limited to this one. As exemplified in sixth and seventh embodiments hereunder, any arrangement may be employed if only the temperature across the outer surface of the end cap 21 can be uniformed by the arrangement.

The surgical treatment devices of sixth and seventh embodiments each have the configuration as the surgical treatment device 11 of the first embodiment. The only difference of the sixth and seventh embodiments from the first embodiment is the arrangement of the heater elements. Thus, in the following description, the constructional elements of the sixth and seventh embodiments having the similar configurations to those of the constructional elements of the first embodiment, are referred to by the same reference numbers, and descriptions therefor are omitted.

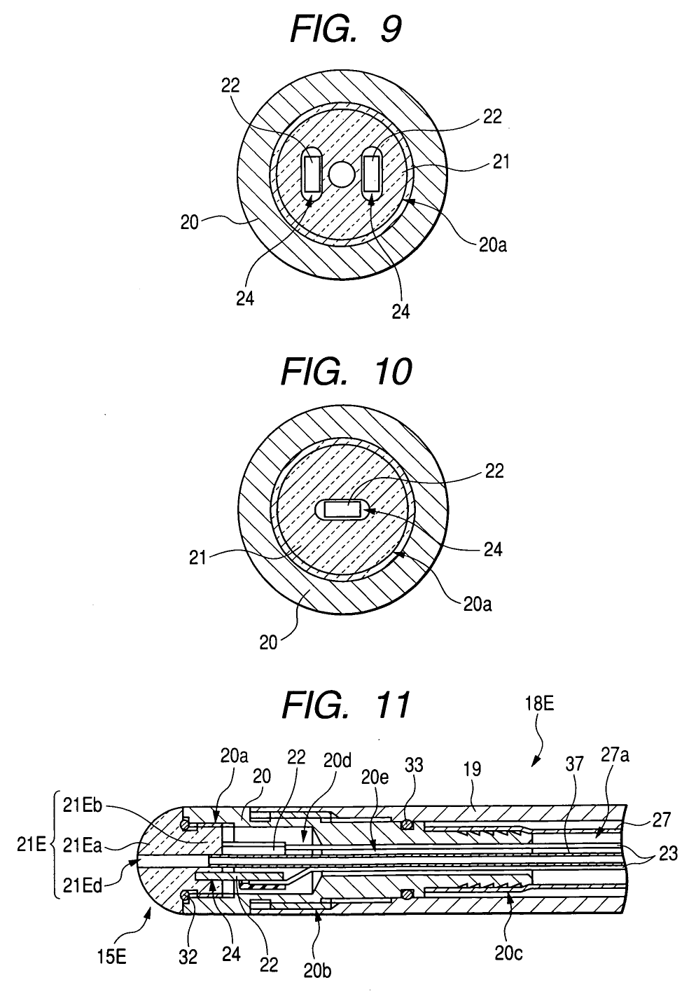

FIG. 9 is a vertical cross-sectional view of a portion in the vicinity of a tip portion (taken along the IV-IV line of FIG. 3) of a surgical treatment device of a sixth embodiment according to the present invention.

The surgical treatment device of the present embodiment includes two heater elements 22 which are disposed so as to face with each other with the longitudinal axis of the treatment device (11; see FIG. 2) being interposed therebetween. The two heater elements 22 are capable of generating quantity of heat sufficient for uniformly heating the end cap 21.

The configuration of the present embodiment is the same as the first embodiment except as described above.

According to the sixth embodiment having the configuration described above, safe and efficient coagulation treatment can be effected as in the first embodiment. Moreover, in the present embodiment, owing to the less number of heater elements 22, i.e. the reduced number of parts, an advantageous effect of reducing manufacturing costs can be provided.

FIG. 10 is a vertical cross-sectional view of a portion in the vicinity of a tip portion (taken along the IV-IV line of FIG. 3) of a surgical treatment device according to a seventh embodiment of the present invention.

The surgical treatment device of the present embodiment includes a single heater element 22 which is disposed on the longitudinal axis of the treatment device (11; see FIG. 2). This single heater element 22 is capable of generating quantity of heat sufficient for uniformly heating the end cap 21.

The configuration of the present embodiment is the same as the first embodiment except as described above.

According to the seventh embodiment having the configuration described as above, the similar effects as in the sixth embodiment can be obtained.

Hereinafter is described a surgical treatment device according to an eighth embodiment of the present invention.

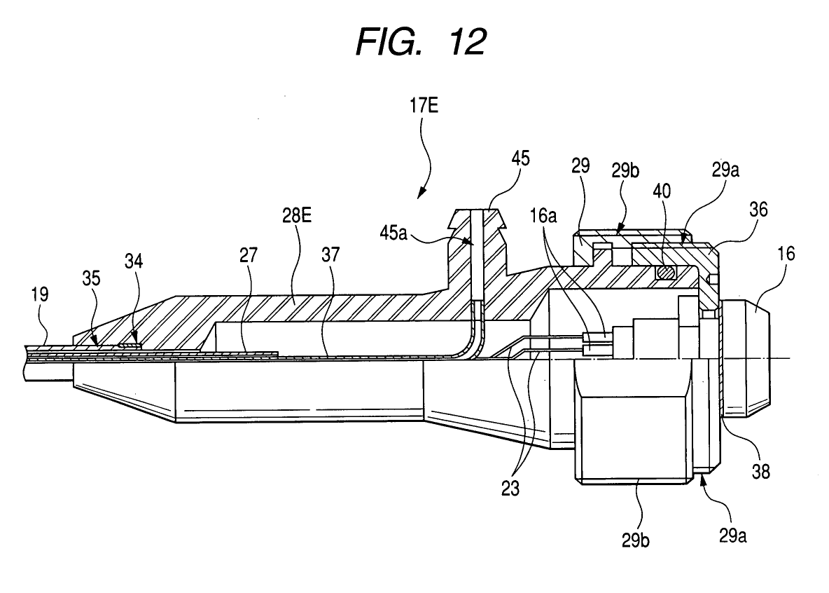

FIG. 11 is a cross-sectional side view showing an inner configuration of a tip portion of a surgical treatment device according to the present embodiment. FIG. 12 is a schematic half cross-sectional side view showing an inner configuration of an operating part of the surgical treatment device according to the present embodiment.

The surgical treatment device of the present embodiment has substantially the same configuration as the surgical treatment device 11 of the first embodiment. As shown in FIG. 11, a through hole 21Ed is provided to an end cap 21E of a treatment portion 15E so as to communicate with the bore of a small diameter tube 37 inserted through a shaft 18E. As a result, the through hole 21Ed is in communication with an air/liquid supply connector 45 provided at a grip member 28E of an operating part 17E, which makes a difference from the first embodiment. Thus, in the following description, the constructional elements having the similar configurations to those of the constructional elements of the first embodiment, are referred to by the same reference numbers, and descriptions therefor are omitted.

As shown in FIG. 11, the end cap 21E of the treatment portion 15E in the surgical treatment device according to the present embodiment is formed of a hemispherical portion 21Ea having a substantially semicircular cross section, and a protrusion 21Eb which is integral with the hemispherical portion 21Ea and protrudes rearward.

In the end cap 21E, the through hole 21Ed is punched through the center of the hemispherical portion 21Ea and through the center of the protrusion 21Eb. One end of the small diameter tube 37 is fitted into this rear end of the through hole 21Ed. The small diameter tube 37 is inserted through the bore of the connecting member 20 and a hollow portion 27a of the tube 27, with its other end being fitted into the air/liquid supply connector 45 provided at the grip member 28E of the operating part 17E, as shown in FIG. 12. The small diameter tube 37 has an airtight or watertight structure.

The airtight or watertight structure of the small diameter tube 37 prevents the leads 23 which are accommodated in the tube 27 together with the small diameter tube 37 and prevents the heater elements 22 disposed in the bore of the large diameter portion 20d of the connecting member 20, from being directly in contact with air or liquid that passes through the small diameter tube 37.

As shown in FIG. 12, the air/liquid supply connector 45 is formed at a portion midway of the grip member 28E of the operating part 17E. As described above the rear end of the small diameter tube 37 is fitted into a bore 45a of the air/liquid supply connector 45.

Is should be noted that a conduit extending, such as from an air/liquid supply pump or a suction pump (not shown) is connected to the air/liquid supply connector 45. Accordingly, the air/liquid supply connector 45 takes the form of a tube joint.

The configuration of the present embodiment is the same as the first embodiment except as described above.

According to the present embodiment, efficient coagulation treatment or blunt separation treatment can be carried out, as in the first embodiment, while suppressing heat loss, by heating the treatment portion 15 with the operation of the heater elements 22.

In the present embodiment, the air/liquid supply connector 45 is provided in the operating part 17E. The air/liquid supply connector 45 is fitted with the small diameter tube 37 which extends from the operating part 17E to the treatment portion 15E through the shaft 18E. Accordingly, air supply operation, liquid supply operation or suction operation for the tissues in a body cavity may be performed, by connecting an air/liquid supply pump, suction pump or the like, as required, to the air/liquid supply connector 45 through the conduit.

As described above, according to the present embodiment, the similar effects as the first embodiment can be obtained. In addition, according to the present embodiment, different treatments (such as an air supply treatment, a liquid supply treatment or a suction treatment) may be effected using the same surgical treatment device.

A surgical treatment device according to a ninth embodiment of the present invention is described below.

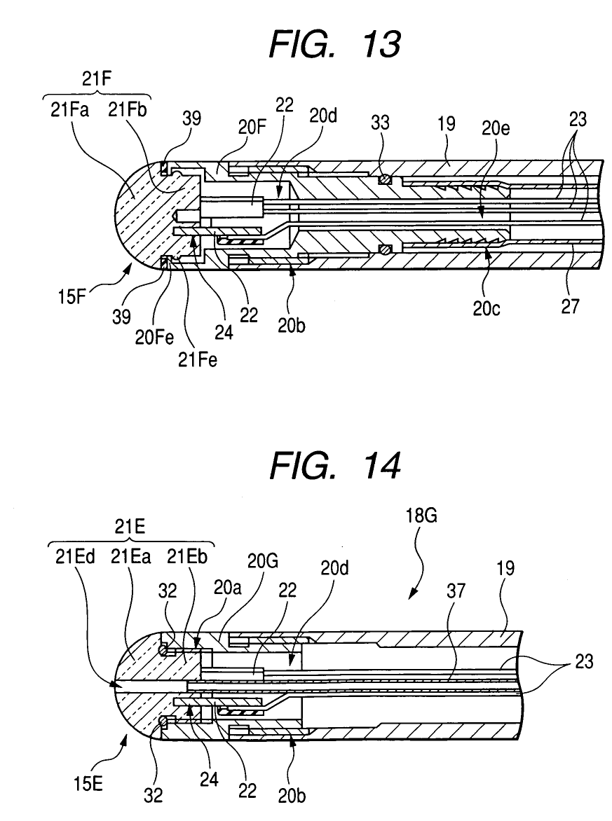

FIG. 13 is a cross-sectional side view showing an inner configuration of a tip portion of a surgical treatment device according to the ninth embodiment of the present invention.

The surgical treatment device of the present embodiment has a configuration which is substantially the same as the surgical treatment device 11 of the first embodiment. As shown in FIG. 13, the present embodiment is different from the first embodiment in that an end cap 21F of a treatment portion 15F is configured to be detachable for a connecting member 20F. Accordingly, in the following description, the constructional elements of the present embodiment having the similar configurations to those of the constructional elements of the first embodiment, are referred to by the same reference numbers, and descriptions therefor are omitted.

As shown in FIG. 13, the end cap 21F of the treatment portion 15F of the surgical treatment device according to the present embodiment is formed of a hemispherical portion 21Fa having a semicircular side cross section, and a protrusion 21Fb which is integral with the hemispherical portion 21Fa and protrudes rearward.

An annular engaging portion 21Fe is integrally formed at the outer peripheral surface of the protrusion 21Fb of the end cap 21F.

Further, a to-be-engaged portion 20Fe of an annular shape is formed, in an inwardly protruding manner, at the inner peripheral surface at an end portion of the connecting member 20F. The to-be-engaged portion 20Fe is engaged with the engaging portion 21Fe.

In the present embodiment, the end cap 21F can be mounted on the connecting member 20F, by inserting the protrusion 21Fb of the end cap 21F into the end side of the connecting member 20F, and pushing the end cap 21F up to a position where the to-be-engaged portion 20Fe is engaged with the engaging portion 21Fe.

The engagement state between the to-be-engaged portion 20Fe and the engaging portion 21Fe is released by only imposing a force, to the end cap 21F, in the direction of pulling out the end cap 21F. Thus, the end cap 21F can be readily removed from the connecting member 20F. In the present embodiment, the end cap 21F is adapted to be detachable for the connecting member 20F.

The present embodiment is configured such that, when the end cap 21F is being attached to the connecting member 20F, a sealing member 39 is interposed between the both. The sealing member 39 ensures a watertight structure between the end cap 21F and the connecting member 20F. The configuration of the present embodiment is the same as the first embodiment except as described above.

As described above, according to the ninth embodiment, the similar effects as in the first embodiment can be obtained. In addition to this, the present embodiment is configured such that the end cap 21F and the connecting member 20F are linked by allowing the engaging portion 21Fe of the end cap 21F to be engaged with the to-be-engaged portion 20Fe of the connecting member 20F to thereby facilitate assembling of the surgical treatment device.

A surgical treatment device according to a tenth embodiment of the present invention is described below.

FIG. 14 is a cross-sectional side view showing an internal configuration of a tip portion of a surgical treatment device according to the tenth embodiment of the present invention.

The surgical treatment device of the present embodiment has substantially the same configuration as the eighth embodiment. The only difference between the present embodiment and the eighth embodiment is the shape of a connecting member 20G which links the end cap 21E of the treatment portion 15E with the tubular member 19 of a shaft 18G. Accordingly, in the following description, the constructional elements having the similar configurations to those of the constructional elements of the eighth embodiment, are referred to by the same reference numbers, and descriptions therefor are omitted.

As shown in FIG. 14, unlike the connecting member 20 in each of the embodiments described above, which has a large diameter portion and a small diameter portion, the connecting member 20G of the surgical treatment device of the present embodiment only has a large diameter portion 20d. This configuration eliminates the use of the tube 27 which has been adapted to be linked with the connecting member 20 in each of the embodiments described above. This configuration is advantageous if the electrical insulation between the leads 23 and the tubular member 19 can be ensured.

In the present embodiment, the protrusion 21Eb of the end cap 21E and the connecting member 20 are linked together through the first engaging portion 20a of the connecting member 20.

As in the eighth embodiment, the through hole 21Ed is punched through the end cap 21E. One end of the small diameter tube 37 is fitted into the through hole 21Ed. The small diameter tube 37 is inserted through the shaft 18G, with its other end being fitted into the air/liquid supply connector of the grip member of the operating part (see FIG. 12).

As described above, in the present embodiment, the small diameter tube 37 and the leads 23 are accommodated in the tubular member 19 of the shaft 18G.

The configuration of the present embodiment is the same as the eighth embodiment except as described above.

According to the tenth embodiment, similar effects as in the eighth embodiment can be obtained as described above. In addition to this, according to the present embodiment, a surgical treatment device having the connecting member 20 of more simplified shape can be provided.

In each of the embodiments described above, there are 3 heater elements at the most, but without limitation to this, more heater elements 22 can be disposed as required if there is spatial room in the end cap.

As a material for the tubular member 19, stainless material is used, for example. Preferred materials for the heat insulating member include, for example, PEEK® (polyetheretherketone), fluoroethylene tetraflouride (known as Teflon®) and polyimide, however, materials of low heat conductivity, such as glass, or thermosetting resins, such as epoxy resins may be used. As a material of the grip member, PEEK® or RADEL® (polyphenylsulfone) may be used.

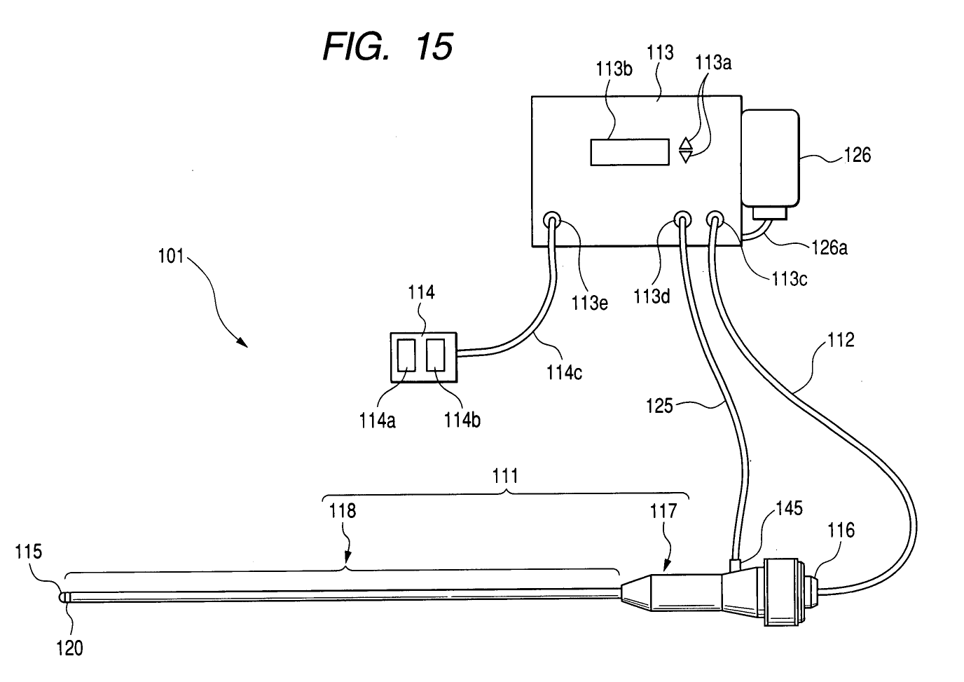

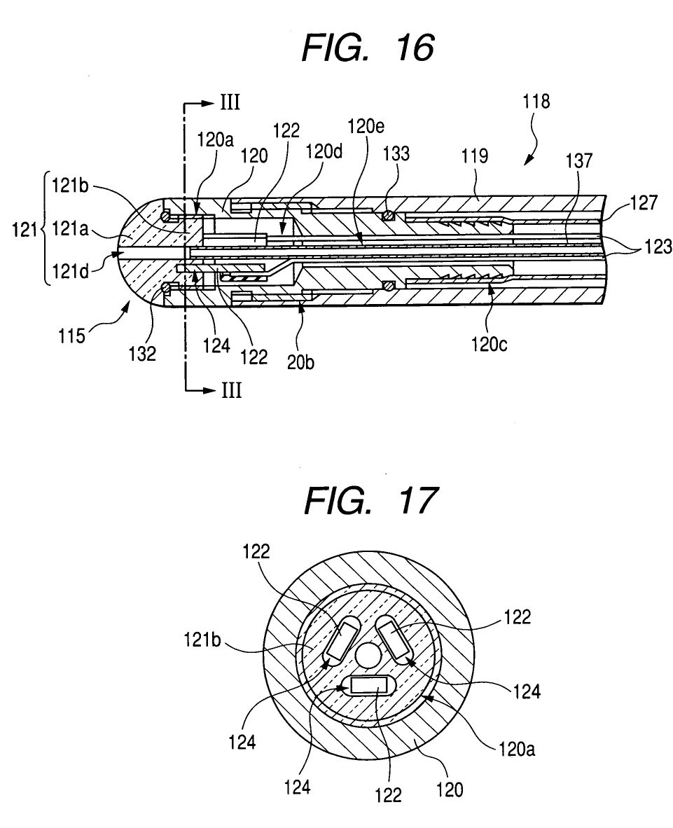

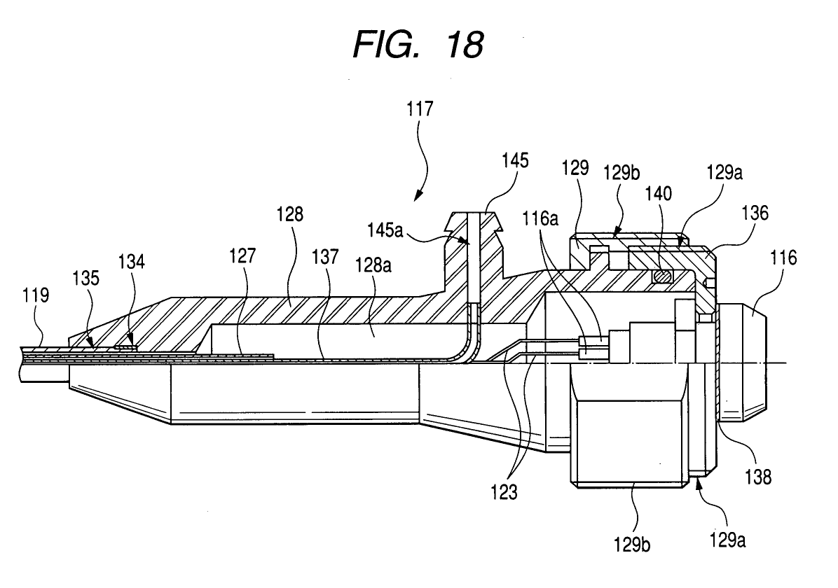

FIG. 15 is a schematic block diagram showing a configuration of a surgical treatment system including a surgical treatment device according to an eleventh embodiment of the present invention. FIG. 16 is a cross-sectional side view of an inner configuration of a tip portion of a surgical treatment device of the present invention. FIG. 17 is a vertical cross sectional view taken along the XVII-XVII line of FIG. 16. FIG. 18 is a schematic half cross-sectional side view of an operating part of the surgical treatment device according to the present embodiment.

A surgical treatment device 111 of the present embodiment is used as a part of a surgical treatment system 101. As shown in FIG. 15, the surgical treatment system 101 mainly includes: the surgical treatment device 111 of the present embodiment; a power supply unit 113 provided therein with a liquid supply pump (not shown), connected to the surgical treatment device 111 through a connecting cable 112 and a liquid supply conduit 125, and supplies power to the whole surgical treatment system 101; a foot switch 114 which is connected to the power supply unit 113 through a cable 114c, and generates a signal for on-off control of a heater element by having a user step thereon, for example; and a liquid supply tank 126 which is detachably attached to the power supply unit 113, and connected to the liquid supply pump in the power supply unit 113 through a conduit 126a, for storage of liquid.

The surgical treatment device 111 mainly includes: an operating part 117 including a portion to be gripped (hereinafter referred to as a grip portion) by a user, a connector 116 (first connector) which is provided at a base end of the grip portion and to which the connecting cable 112 is connected, and a liquid supply connector 145 (second connector) to which a liquid supply conduit 125 (will be described later) provided at the side of the grip is connected; a treatment portion 115 which is provided at the tip of the surgical treatment device 111 and effects heat treatment to living tissues; and a shaft 118 for linking the treatment portion 115 with the operating part 117.

The shaft 118, as shown in FIG. 16, includes: a tubular member 119 which serves as a mantle tube for protecting leads 123 (cable members), which will be described later; and a connecting member 120 which is disposed between the tubular member 119 (mantle tube) and the treatment portion 115, for linkage therebetween. A tube 127, which is formed of an insulating material, is inserted through the tubular member 119 so as to extend from the rear end of the connecting member 120 to the operating part 117. In the tube 127 (i.e. in the shaft 118), leads 123, which extend from the rear ends of respective heater elements 122 (will be described later) in the treatment portion 115, are accommodated. Each one end of the leads 123 is connected to each of connecting terminals 116a (see FIG. 18) of the connector 116 of the operating part 117.

The power supply unit 113 has a function of controlling the heat generating performance of the heater elements 122 (see FIG. 16) provided in the treatment portion 115 of the surgical treatment device 111. Specifically, as shown in FIG. 15, a temperature setting button 113a for setting temperature of the treatment portion 115, and a display 113b for indicating a temperature value or level set through the temperature setting button 113a, are disposed on the front panel of the power supply unit 113. By operating the temperature setting button 113a, a user can set the temperature of the treatment portion 115 at any level. The power supply unit 113 carries out control so that the temperature of the treatment portion 115 is maintained at a set value.

The front panel of the power supply unit 113 is further provided with an electric terminal 113c to which one end of the connecting cable 112 is connected, a liquid supply connector 113d to which one end of the liquid supply conduit 125 is connected, and a switch terminal 113e to which one end of a cable 114c extending from the foot switch 114 is connected.

The liquid supply connector 113d is connected to the liquid supply pump in the power supply unit 113 through a conduit (not shown).

As described above, the treatment portion 115 is provided at the tip of the shaft 118. The treatment portion 115 includes, as shown in FIG. 16, an end cap 121, the heater elements 122 which are disposed in the end cap 121 and whose resistance change depending on temperature.

The foot switch 114 includes a first pedal 114a for producing a signal for on-off control of power supply to the heater elements 122 from the power supply unit 113, and a second pedal 114b for producing a signal for on-off control of the liquid supply performance of the liquid supply pump disposed in the power supply unit 113.

The end cap 121 is formed of a material having high conductivity, such as copper, molybdenum and platinum. Further, the outer surface of the end cap 121 is given with surface treatment, e.g. Teflon coating® which can inhibit clinging of tissues when used in a body cavity.

The end cap 121 is formed of a hemispherical portion 121a having a semicircular side cross section, and a protrusion 121b which is integral with the hemispherical portion 121a and protrudes rearward. In the end cap 121, a through hole 121d is punched through the center of the hemispherical portion 121a and the center of the protrusion 121b.

A plurality of grooves 124 having a predetermined depth are formed on one circumference at the protrusion 121b of the end cap 121, with a predetermined interval therebetween. Each of the heater elements 122 is fixed to each of these grooves 124 by soldering and brazing, for example. Thus, the heater elements 122 are thermally coupled to the end cap 121.

The outer peripheral portion of the protrusion 121b of the end cap 121 is engaged with a first engaging portion 120a of the connecting member 120. A second engaging portion 120b which is formed on the outer peripheral surface at a portion midway of the connecting member 120, is engaged with the inner peripheral surface of a tip side portion of the tubular member 119. Thus, the end cap 121 and the tubular member 119 (i.e. the shaft 118) are linked with each other through the connecting member 120. An O-ring 132 formed of a waterproof member is provided at a connecting portion between the end cap 121 and the connecting member 120. Further, an O-ring 133 formed of a waterproof member is provided at a connecting portion between the connecting member 120 and the tubular member 119. Thus, watertightness is ensured at each of the connecting portions.

The connecting member 120 is an annular member formed of a material having low heat conductivity with electrical insulating properties. As described above, the connecting member 120 includes: the first engaging portion 120a which is formed at the inner peripheral surface of its tip portion and is engaged with a predetermined portion of the end cap 121 (the outer peripheral portion of the protrusion 121b); the second engaging portion 120b which is formed at the outer peripheral surface at a portion midway thereof and is engaged with a predetermined portion of the tubular member 119 (the inner peripheral surface of the tip portion); and a tube joint 120c which is formed at the outer peripheral surface of its rear end portion and is linked with the tube 127.

One end of the tube 127 is linked to the tube joint 120c at the rear end of the connecting member 120. The tube 127 is inserted through the bore of the tubular member 119 of the shaft 118, with the other end thereof being fixed to a predetermined portion in the operating part 117.

In the bore of the connecting member 120, a large diameter portion 120d is formed at the tip side, and a small diameter portion 120e is formed at the rear side. In the bore of the large diameter portion 120d, the heater elements 122 are partially disposed. The leads 123 which extend from the heater elements 122 are inserted through the bore of the small diameter portion 120e which communicates with the bore of the large diameter portion 120d. As described above, the leads 123 are inserted through the bore of the tube 127 and connected, respectively, to connecting terminals 116a (see FIG. 18) of the connector 116.

As shown in FIG. 15, the connecting cable 112 is connected to the connector 116, by which an electrical connection between the heater elements 122 and the power supply unit 113 is ensured. In other words, the heater elements 122 are electrically connected to the power supply unit 113 through the leads 123, the connector 116 and the connecting cable 112.

As shown in FIG. 17, the plurality (three in the present embodiment) of heater elements 122 are disposed on one circumference in the protrusion 121b of the end cap 121, with a certain interval therebetween. Thus, the entire end cap 121 can be uniformly heated. The number of the heater elements 122 can be adjusted depending on the heat capacity of the end cap 121.

When three heater elements 122 are disposed as described above, the portion surrounded by these heater elements 122 creates a dead space. In the present embodiment, the through hole 121d is formed through the dead space, so that a bore structure is realized extending from the treatment portion 115 to the operating part 117 through the shaft 118 without increasing the diameter of the shaft 118.

One end of a small-diameter tube 137 is fitted into the rear end of the through hole 121d of the end cap 121. The small-diameter tube 137 is inserted through bores 120d and 120e of the connecting member 120, and through a hollow portion 127a of the tube 127, i.e. the shaft 118, with its other end being fitted into a bore 145a of a liquid supply connector 145 which is provided at a given portion at the side of a grip member 128 (see FIG. 18), which will be described later, of the operating part 117. The small-diameter tube 137 has a watertight structure through which liquid can pass through.

The watertight structure of the small-diameter tube 137 prevents the leads 123 and the heater elements 122 accommodated in the tube 127 together with the small-diameter tube 137, from being in contact with liquid inside the small-diameter tube 137.

The operating part 117, as shown in FIG. 18, is mainly formed of the grip member 128 gripped by a user, a clamp member ˜129 provided at the base side of the grip member 128, and a connector fixing member 136 for fixing the connector 116 by being engaged with the clamp member 129.

The rear end side portion of the tubular member 119 is fixed to the end side portion of the hollow portion 128a of the grip member 128. The grip member 128 and the tubular member 119 are fixed with each other, at their connecting portion, by a locking member 134 and an adhesive filler material 135.

As described above, the tube 127 is inserted through the tubular member 119. The rear end of the tube 127 is positioned midway of the hollow portion 128a of the grip member 128. The leads 123 and the small-diameter tube 137 are inserted through the tube 127.

The leads 123 are connected to the connecting terminals 116a of the connector 116, in the hollow portion 128a of the grip member 128, so that the leads 123 are sufficiently protected from the outside impulse.

The small-diameter tube 137 further extends out rearward from the tube 127. The rear end of the small-diameter tube 137 is fitted into the bore 145a of the liquid supply connector 145, in the hollow portion 128a of the grip member 128.

The clamp member 129 is disposed at the outer periphery of the rear end side portion of the grip member 128. An engaging portion 129a is formed at the inner periphery of the rear end side portion of the clamp member 129. The connector fixing member 136 is fitted into the engaging portion 129a to fix the connector 116. The connector fixing member 136 is disposed between the engaging portion 129a of the clamp member 129 and the outer peripheral surface of the rear end side portion of the grip member 128. An O-ring 140 formed of a waterproof member is provided between the grip member 128 and the connector fixing member 136. Further, a seal member 138 having waterproof properties is provided between the connector fixing member 136 and the connector 116.

An engaging portion 129b is formed at the outer peripheral surface of the clamp member 129. A protection cover (not shown) for protecting the connector 116 is engaged with the engaging portion 129b. By attaching the protection cover, as required, using the engaging portion 129b of the clamp member 129, the surgical treatment device 111 can be dipped in an antiseptic chemical, for example.

The liquid supply connector 145 is formed integrally with the grip member 128 at a portion half way down the grip member 128. The liquid supply connector 145 has a tube joint shape, so that the liquid supply conduit 125 connected to the liquid supply connector 113d of the power supply unit 113 at one end thereof can be connected to the liquid supply connector 145 at the other end thereof.

As described above, the surgical treatment system including the surgical treatment device 111 according to the present embodiment is provided with liquid supply means which includes the liquid tank 126, the liquid supply pump disposed inside the power supply unit 113, the conduit 126a connecting the liquid supply pump and the liquid supply tank 126, the liquid supply connector 145 at the operating part 117 of the surgical treatment device 111, the liquid supply conduit 125 connecting the liquid supply connector 145 and the liquid supply pump (power supply unit 113), and the small-diameter tube 137 inserted through the surgical treatment device 111.

The operation of the surgical treatment system having the configuration described above will be described below.

First of all, a user can connect the surgical treatment device 111 and the foot switch 114 to the power supply unit 113. Thereafter, a start-up preparation can be carried out, including turning on the power supply unit 113.

After making the surgical treatment system of the present embodiment be ready for use, a user can operate the temperature setting button 113a of the power supply unit 113 to set the temperature of the treatment portion 115 at an optimum level for the treatment to be given. In this case, the result of setting, namely set temperature (or set level) is indicated on the display 113b.

Then, a user can hold the operating part 117 of the surgical treatment device 111, and then can allow the treatment portion 115 of the surgical treatment device 111 to be in contact with tissues, followed by stepping on the first pedal 114a of the foot switch 114. Thus, electrical power is supplied to the heater elements 122 from the power supply unit 113 to permit the heater elements 122 to generate heat.

The heater elements 122, as described above, are characteristic in that their resistances are varied depending on temperature. By monitoring the resistance of the heater elements 122, the power unit 113 can control the temperature of the heater elements 122 so as to be kept at a level preset by the user.

Heat generated by the heater elements 122 is transferred to the end cap 121, by which the treatment portion 115 is heated. The heater elements 122 are directly coupled to the end cap 121 as described above. Therefore, no heat loss is caused in the transfer of heat from the heater elements 122 to the end cap 121. Thus, the temperature of the treatment portion 115 becomes approximately the same as the preset temperature.

The heater elements 122 are disposed on one circumference in the end cap 121 with approximately an even interval therebetween. The number and positions of the heater elements 122 can be changed depending on the heat capacity of the end cap 121. The end cap 121 is formed of a material having high heat conductivity, while the connecting member 120 to be linked to the treatment portion 115 is formed of a material having low heat conductivity. In this way, the treatment portion 115 is thermally insulated from outside, so that the temperature of the outer surface of the treatment portion 115 becomes approximately even. Thus, the same effects can be given to the tissues by any portion on the outer surface of the treatment portion 115, which is allowed to be in contact with the tissues in a body cavity.

As is apparent from the description provided above, the connecting member 120, being disposed between the treatment portion 115 having the heater elements 122 and the tubular member 119 (mantle tube), serves as a linkage between the both, and at the same time, serves as insulating means for inhibiting heat transfer between the both.

As described above, surface treatment, such as Teflon coating® is applied to the outer surface of the end cap 121. Meanwhile, the connecting member 120 is formed of a material having low heat conductivity. For these reasons, the temperature of the connecting member 120 is not so increased as the treatment portion 115. Accordingly, when the tissues of a body cavity are in contact with the outer surfaces of the end cap 121 and the connecting member 120, clinging of tissues onto the outer surfaces is suppressed.

Furthermore, the treatment portion 115, the connecting member 120, the shaft 118 and the operating part 117 are integrally linked, and configured to have a shape of a rigid rod. Thus, when a user holds and moves the operating part 117, the treatment portion 115 moves accordingly. In this way, a user can readily move the treatment portion 115 to a target position by operating the operating part 117 to thereby efficiently carry out blunt separation of tissues.

When a user steps on the foot switch 114 during the operation as described above, blunt separation of the tissues can be efficiently performed while giving coagulation treatment by the treatment portion 115.

Further, when a user steps on the second pedal 114b of the foot switch 114, the liquid supply pump in the power supply unit 113 is operated, whereby the liquid stored in the liquid supply tank 126 is supplied therefrom to the liquid supply pump, passing through the conduit 126a. After that, the liquid passes through the liquid supply conduit 125, passes through the small-diameter tube 137 inserted through the operating part 117 and the shaft 118, further passes through the through hole 121d of the end cap 121 of the treatment portion 115, and is discharged outward from the opening at the tip. Tissues are washed by the discharged liquid. In this way, confirmation of a bleeding point and cooling of tissues after cautery can be carried out.

As described above, according to the eleventh embodiment, coagulation treatment and blunt separation treatment can be safely and efficiently carried out. In addition, a user can carry out liquid supply operation after treatment without changing devices to thereby shorten operation time.

The eleventh embodiment enables both treatment and liquid supply simultaneously because the treatment portion 115 equipped with the heater elements 122 is independent of the liquid supply means.

Further, since the through hole 121d formed in the end cap 121 extends in a direction parallel to the longitudinal axes of the treatment portion 115 and the shaft 118, and since liquid is also discharged in this direction from the opening of the through hole 121d, liquid can be readily supplied to tissues under treatment and to the vicinity of the tissues during treatment or between treatments, for example.

Moreover, when the opening of the through hole 121d is blocked, such as by tissues attached to the outer surface of the treatment portion 115, for example, the opening provided to the treatment portion 115 readily enables removal of the attachment by the liquid supply operation.

In addition, since the through hole 121d is formed in the dead space (portion surrounded by the heater elements) in the treatment portion 115, the diameter of the shaft 118 can be made smaller.

Although the tubular member 119 of the shaft 118 has been described as having rigidity in the eleventh embodiment, without limitation to this, the tubular member 119 may be formed of a material which can be curved or bent.

In the eleventh embodiment, the heater elements 122 are directly fixed, by soldering or brazing, for example, to the respective grooves 124 formed in the protrusion 121b of the end cap 121 to thereby establish direct thermal coupling between the heater elements 122 and the end cap 121, however, the manner of fixing the heater elements 122 is not limited to this. For example, the heater elements 122 may be linked to the end cap 121 through a member having good heat conductivity.

In the eleventh embodiment, although the liquid supply pump is disposed in the power supply unit 113, the liquid supply pump may be detachable with respect to the power supply unit 113.

A surgical treatment system according to a twelfth embodiment of the invention is described hereunder.

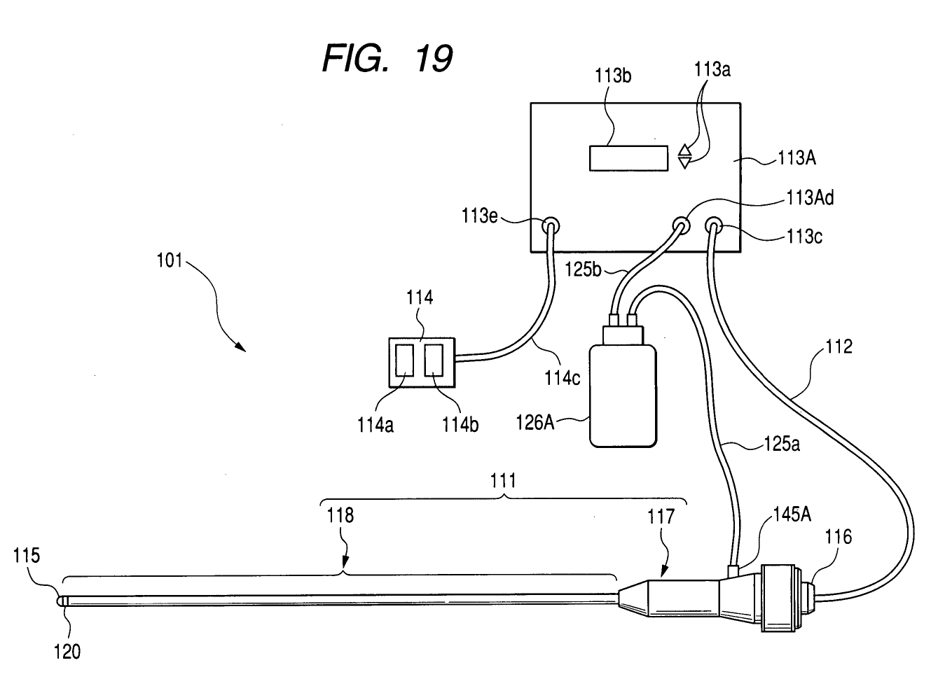

FIG. 19 is a schematic block diagram of the surgical treatment system according to the twelfth embodiment of the present invention.

The surgical treatment system of the present embodiment has substantially the same configuration as the eleventh embodiment. The present embodiment is different from the eleventh embodiment in that the present embodiment has suction means instead of the liquid supply means. Accordingly, in the following description, the constructional elements of the present embodiment having the similar configurations to those of the constructional elements of the eleventh embodiment are referred to by the same reference numbers, and descriptions therefor are omitted.

In the present embodiment, the small-diameter tube 137 (see FIG. 16) is inserted through the tube 127 as in the eleventh embodiment. Instead of the liquid supply connector 145 (see FIG. 15) of the eleventh embodiment, a suction connector 145A (second connector) of the same shape is disposed at the operating part 117 of the surgical treatment device 111.

Further, instead of the liquid supply pump in the eleventh embodiment, a suction pump (not shown) is disposed in a power supply unit 113A. Also, instead of the liquid supply tank 126 (see FIG. 15) in the eleventh embodiment, a suction bottle 126A is disposed separately from the power supply unit 113A.

The suction connector 145A of the operating part 117 and the suction bottle 126A are connected through a first suction conduit 125a. The suction bottle 126A and the power supply unit 113A are connected through a second suction conduit 125b. A suction connector 113Ad, to which one end of the second suction conduit 125b is connected, is provided at the front panel of the power supply unit 113A. The suction connector 113Ad is connected to the suction pump inside the power supply unit 113A.

The foot switch 114 of the present embodiment includes the two pedals 114a and 114b. As in the eleventh embodiment, the first pedal 114a is for allowing the treatment portion 115 to perform heating operation. The second pedal 114b, unlike the eleventh embodiment, is for allowing the suction pump provided in the power supply unit 113A to perform suction operation.

As described above, the present embodiment includes suction means including the suction bottle 126A, the suction pump disposed in the power supply unit 113A, the second suction conduit 125b connecting between suction pump and the suction bottle 126A, the suction connector 145A provided at the operating part 117 of the surgical treatment device 111, the suction conduit 125a connecting between the suction connector 145A and the suction pump disposed in the power supply unit 113A, and the small-diameter tube 137 inserted through the surgical treatment device 111.

The configuration of the present invention is the same as the eleventh embodiment except as described above. The operation of the present embodiment is also substantially the same as the eleventh embodiment, the difference from the eleventh embodiment being the performance effected by the operation of the second pedal 114b.

Specifically, in the present embodiment, when a user steps on the second pedal 114b as required, the suction pump provided in the power supply unit 113 is operated, so that a suction pressure is imposed inside the small-diameter tube 137. In response to this, materials, such as liquid or gases, are sucked inward, from the vicinity of tissues in a body cavity, through the opening of the through hole 121d of the end cap 121 of the treatment portion 115. The sucked materials are collected to the suction bottle 126A, passing through the small-diameter tube 137, and through the suction conduit 125a connected to the suction connector 145A.

As described above, according to the twelfth embodiment, coagulation treatment and blunt separation treatment can be efficiently carried out as in the eleventh embodiment. In addition, according to the present embodiment, suction operation after treatment can be carried out without changing devices. Thus, according to the present embodiment, reduction of operation time can be realized.

Alternatively, the present embodiment may be configured such that, on stepping on the first pedal 114a of the foot switch 114, power is supplied to the heater elements 122, and that, at the same time, the suction pump is driven. This configuration enables suction, such as of blood and smoke in the vicinity of tissues subjected to treatment to ensure good visual field, while enabling a user to effect treatment to the target site to be treated by bringing the treatment portion 115 close to the site.

A surgical treatment system according to a thirteenth embodiment of the present invention is described hereunder.

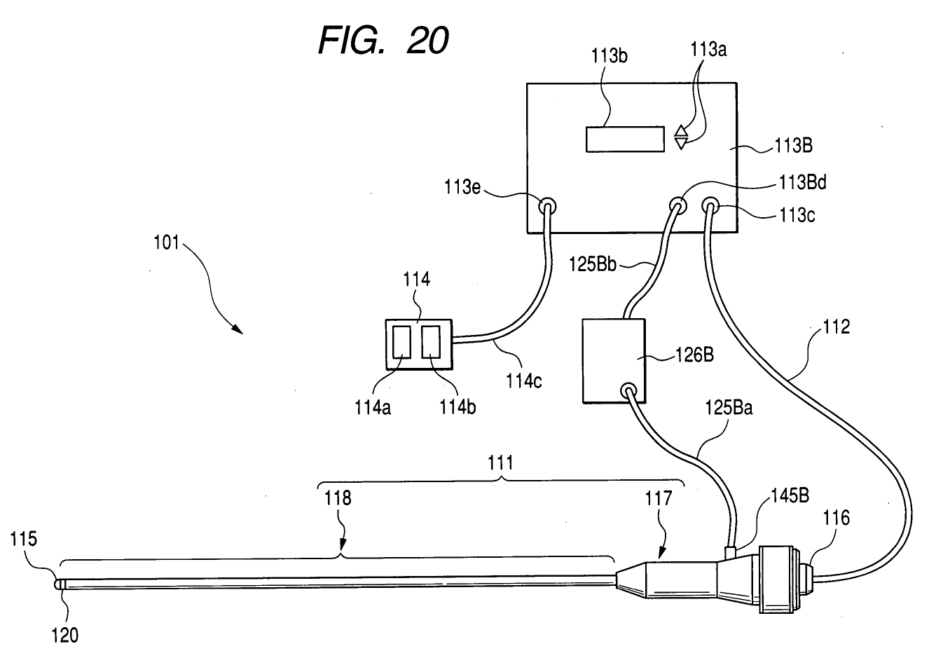

FIG. 20 is a schematic block diagram showing a configuration of the surgical treatment system according to the thirteenth embodiment of the present invention.

The present embodiment has substantially the same configuration as the eleventh embodiment. The present embodiment is different from the eleventh embodiment in that the present embodiment includes air supply means instead of the liquid supply means. Accordingly, in the following description, the constructional elements of the present embodiment having similar configurations to those of the constructional elements of the eleventh embodiment are referred to by the same reference numbers, and descriptions therefor are omitted.

In the present embodiment, the small-diameter tube (137, not shown in FIG. 20) is inserted through the tube 127 as in the eleventh embodiment. Instead of the liquid supply connector 145 (see FIG. 15), a connector 145B (second connector) of the same shape is disposed at the operating part 117 of the surgical treatment device 111.

In addition, in the present embodiment, an air compressor 126B separate from the power supply unit 113B is used instead of the liquid supply pump (built in a power supply unit 113B) and the liquid supply tank 126 (detachably attached to the power supply unit 113B). The air compressor 126B and the power supply unit 113B are electrically connected through a connecting cable 125Bb for supplying power, a control signal or the like to the air compressor 126B. The air compressor 126B and the air supply connector 145B of the operating part 117 are connected through an air supply conduit 125Ba. A connector 113Bd, to which one end of the connecting cable 125Bb is connected, is provided at the front panel of the power supply unit 113B.

The foot switch 114 of the present embodiment includes the two pedals 114a and 114b. As in the eleventh embodiment, the first pedal 114a is for allowing the treatment portion 115 to perform heating operation. The second pedal 114b, unlike the eleventh embodiment, is for allowing the air compressor 126B to perform air supply operation.

In this way, the present embodiment includes suction means which includes the air compressor 126B, the air supply connector 145B provided at the operating part 117 of the surgical treatment device 111, the air supply conduit 125Ba for connecting between the air supply connector 145B and the air compressor 126B, the small-diameter tube 137 inserted through the surgical treatment device 111, and the connecting cable 125Bb for electrically connecting between the air compressor 126B and the connector 113Bd of the power supply unit 113B.

The configuration of the present embodiment is the same as the eleventh embodiment except as described above. Also, the operation of the present embodiment is substantially the same as the eleventh embodiment, but is different from the eleventh embodiment in the performance at the time the second pedal 114b is operated.

Specifically, in the present embodiment, upon user's stepping on the second pedal 114b of the foot switch 114 as required, the air compressor 126B is operated, so that high-pressure air is supplied into the small-diameter tube 137. The high-pressure air is then discharged outward from the opening of the through hole 121d of the end cap 121 of the treatment portion 115. Thus, materials, such as liquid or gases in the vicinity of the tissues to be treated in a body cavity can be removed to thereby ensure good visual field.

As described above, according to the thirteenth embodiment, coagulation treatment and blunt separation treatment of tissues can be efficiently carried out as in the eleventh embodiment. In addition, according to the present embodiment, an air supply operation after treatment can be carried out without changing devices. Accordingly, operation time can be shortened.

In the thirteenth embodiment, although the air compressor 126B is provided separately from the power supply unit 113B, the air compressor 126B may be disposed inside the power supply unit 113B.

A surgical treatment system according to a fourteenth embodiment of the present invention is described hereunder.

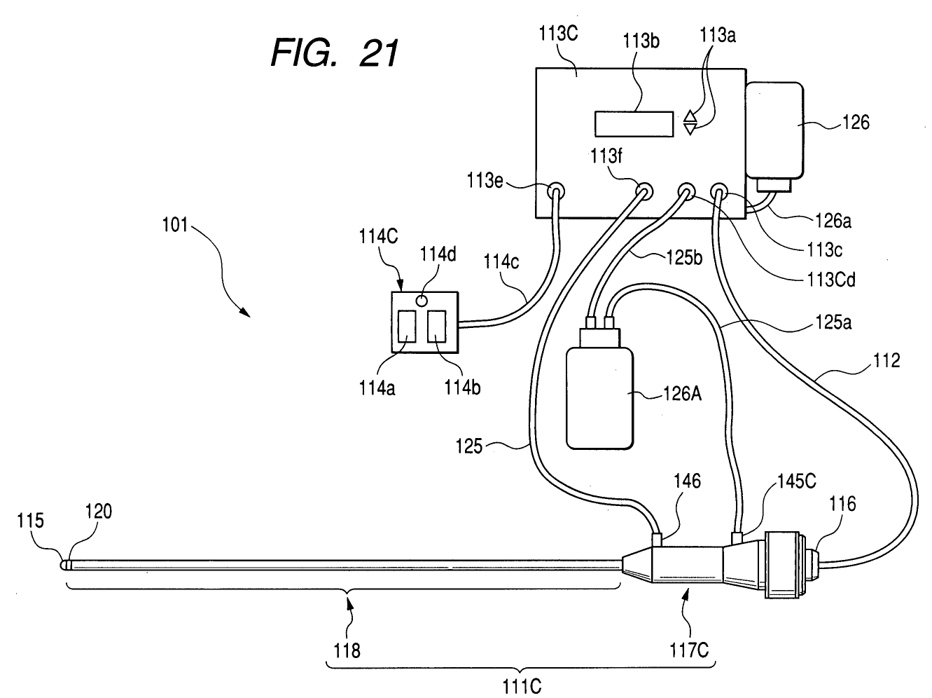

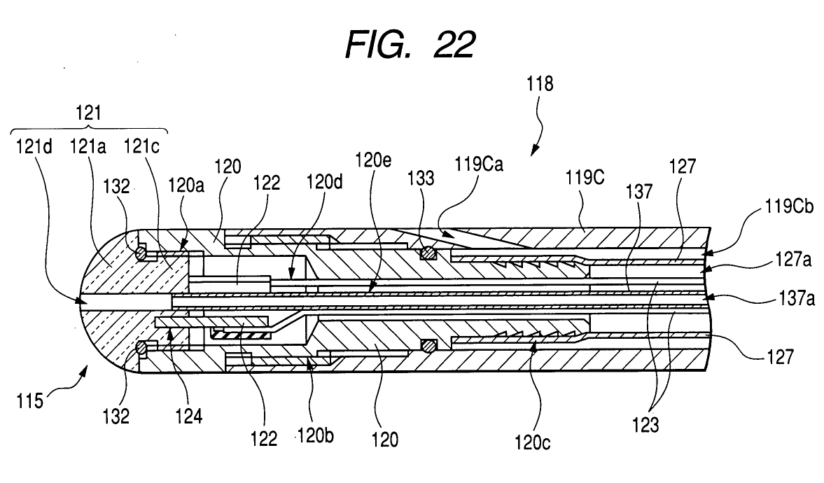

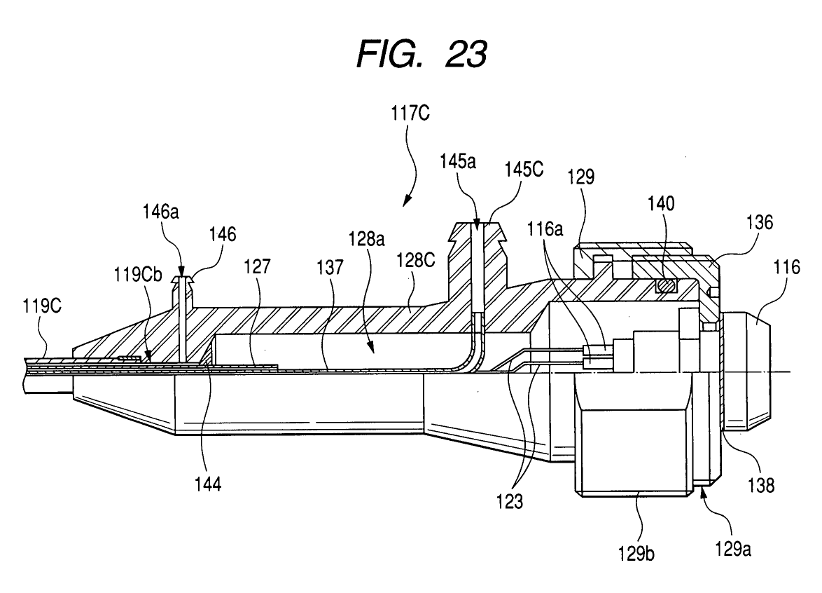

FIG. 21 is a schematic block diagram showing the configuration of the surgical treatment system according to the fourteenth embodiment of the present invention. FIG. 22 is a cross-sectional side view showing the inner configuration of a tip portion of a surgical treatment device according to the present embodiment. FIG. 23 is a schematic half cross-sectional side view showing the inner configuration of an operating part of the surgical treatment device according to the present embodiment.

The present embodiment has substantially the same configuration as the eleventh and the twelfth embodiments. The present embodiment is characteristic in that the liquid supply means in the eleventh embodiment and the suction means in the twelfth embodiment are combined. Thus, in the following description, the constructional elements having the similar configurations to those of the constructional elements of the eleventh and the twelfth embodiments are referred to by the same reference numbers, and descriptions therefor are omitted.

In a power supply unit 113C of the present embodiment, the liquid supply pump (not shown) of the eleventh embodiment and the suction pump (not shown) of the twelfth embodiment are provided.

Also, as in the eleventh embodiment, the liquid supply tank 126 is detachably attached to the power supply unit 113C. The liquid supply pump and the liquid supply tank 126 are connected to each other by a conduit 126a.

The liquid supply pump is connected to a liquid supply connector 113f provided at the front panel of the power supply unit 113C through an inner conduit (not shown) in the power supply unit 113C. One end of the liquid supply conduit 125 is connected to the liquid supply connector 113f, with the other end being connected to a liquid supply connector 146 (third connector) of a surgical treatment device 111C, so that inner communication is established throughout the liquid supply tank 126, the conduit 126a, the liquid supply pump, the liquid supply connector 113f, the liquid supply conduit 125, the liquid supply connector 146, and an operating part 117C of the surgical treatment device 111C.

In the present embodiment, as in the twelfth embodiment, the suction bottle 126A is provided separately from the power supply unit 113C. The suction bottle 126A and the suction connector 145C (second connector) of the operating part 117C are connected to each other through the first suction conduit 125a. The suction bottle 126A and the power supply unit 113C are connected to each other through the second suction conduit 125b. A suction connector 113Cd, to which one end of the second suction conduit 125b is connected, is provided at the front panel of the power supply unit 113C. The suction connector 113Cd is connected to the suction pump inside the power supply unit 113C.

A foot switch 114C is electrically connected to the switch terminal 113e of the power supply unit 113C through the cable 114c. The foot switch 114C includes the first pedal 114a for generating an on-off control signal for supplying power to the treatment portion 115, the second pedal 114b for generating an on-off control signal for operating the liquid supply pump or the suction pump provided inside the power supply unit 113C, and a third pedal 114d for switching the function of the second pedal 114b.

As shown in FIG. 22, the through hole 121d is provided, as in the eleventh embodiment, in the end cap 121 of the shaft 118 of the surgical treatment device 111C according to the present embodiment. One end of the small-diameter tube 137 is fitted into the rear end of the through hole 121d. The small-diameter 137 is inserted through the bores 120d and 120e of the connecting member 120, and through the hollow portion 127a of the tube 127, with its other end being fitted into the bore 145a of the suction connector 145C which is provided at a predetermined position at the side of the grip member 128 (see FIG. 23) of the operating part 117C. As a result, blood, smoke or the like in the vicinity of the tissues subjected to treatment is sucked from the tip of the treatment portion 115 and collected to the suction bottle 126A, passing through a hollow portion 137a of the small-diameter tube 137.

One end of the tube 127 is linked with the tube joint 120c at the rear end of the connecting member 120. The tube 127 is inserted through the bore of a tubular member 119C of the shaft 118, with the other end being fixed to a predetermined position inside the operating part 117C. A gap 119Cb is formed in a space surrounded by the tubular member 119C, the tube 127 and the connecting member 120. Specifically, the gap 119Cb is formed throughout the shaft 118 to form a conduit that communicates with a bore 146a of the liquid supply connector 146 of the operating part 117C. A seal member 144 is provided between the termination of the conduit, i.e. the gap 119Cb and the hollow portion 128a of the grip member 128C, so that the conduit would not communicate with the hollow portion 128a to thereby ensure watertightness of the gap 119Cb.

In the tubular member 119C, a nozzle 119Ca which extends obliquely forward is formed in the vicinity of the connecting portion between the connecting member 120 and the tube 127. Thus, communication is established throughout the liquid supply tank 126, the conduit 126a, the liquid supply pump, the liquid supply conduit 125, the liquid supply connector 146, the gap 119Cb between the tube 127 and the tubular member 119C inside the operating part 117C of the surgical treatment device 111C, and the nozzle 119Ca, so that the liquid in the liquid supply tank 126 can be discharged from the nozzle 119Ca toward the vicinity of the tip of the treatment portion 115. It should be noted that a seal member, for example, which prevents entry such as of liquid may be provided between the tube 127 and the tube joint 120c at the rear end side of the connecting member 120.

As shown in FIG. 23, the suction connector 145C is provided at the rear end side portion of the outer peripheral surface of the operating part 117C of the surgical treatment device 111C, and the liquid supply connector 146 is provided at the front side portion thereof. The small-diameter 137 is connected to the suction connector 145C, while the liquid supply connector 146 communicates with the gap 119Cb between the tube 127 and the tubular member 119C.

The configuration of the present embodiment is the same as the eleventh and the twelfth embodiments except as described above. Also, the operation of the present embodiment is the same as the eleventh and the twelfth embodiments.

As described above, according to the fourteenth embodiment, coagulation treatment and blunt separation treatment of tissues can be efficiently carried out as in the eleventh and the twelfth embodiments. In addition, according to the present embodiment, liquid supply operation or suction operation after treatment can be carried out without changing devices. Accordingly, operation time can be shortened.

In the fourteenth embodiment, the function of the second pedal 114b of the foot switch 114C is switched between liquid supply operation and suction operation by the third pedal 114c, however, the second pedal 114b and the third pedal 114c may be fixedly assigned their respective functions.

For example, the function of generating the on-off control signal for operating the liquid supply pump may be assigned to the second pedal 114b, and the function of generating the on-off control signal for operating the suction pump may be assigned to the third pedal 114c, or viceversa.

In addition, an air compressor similar to that of the thirteenth embodiment, for example, may be connected to the surgical treatment device 111C of the present embodiment. In this case, a circuit for controlling the compressor may be provided to the power supply unit 113C.

In each of the embodiments described above, three heater elements 122 are disposed on the same circumference with a certain interval therebetween, however, the heater elements disposition is not limited to this. For example, as described in a fifteenth embodiment hereunder, any disposition may be adopted if it can provide an approximately uniformed temperature throughout the outer surface of the end cap.

A surgical treatment device according to the fifteenth embodiment includes substantially the same configuration as the surgical treatment device 111 of the eleventh embodiment. In the present embodiment, the only difference from the eleventh embodiment is the disposition of the heater elements 122. Thus, in the following description, the constructional elements having the similar configurations to those of the constructional elements of the eleventh embodiment are referred to by the same reference numbers, and descriptions therefor are omitted.

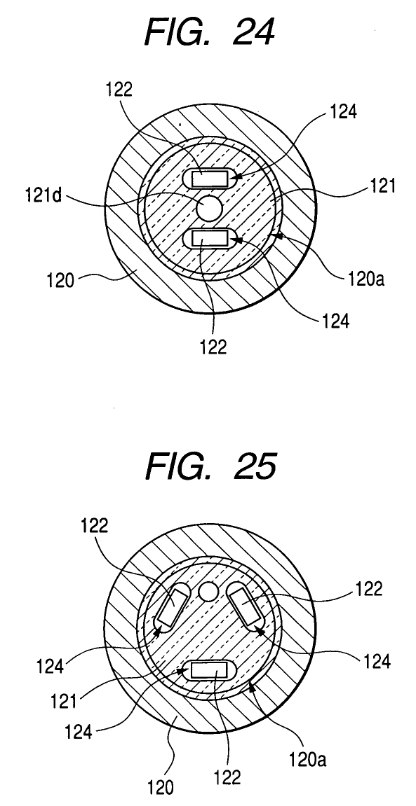

FIG. 24 is a vertical cross-sectional view at a portion (along the XVII-XVII line of FIG. 16) in the vicinity of a tip portion of the surgical treatment device according to the fifteenth embodiment of the present invention.

The surgical treatment device according to the present embodiment includes two heater elements 122. The two heater treatments 122 are disposed so as to face with each other being interposed by a longitudinal axis of the treatment device (111, see FIG. 15). These two heater elements are capable of generating a heat quantity sufficient for uniformly heating the end cap 121.

The configuration of the present embodiment is the same as the eleventh embodiment except as described above.

According to the fifteenth embodiment having the configuration as described above, coagulation treatment can be safely and efficiently carried out as in the eleventh embodiment. In addition, according to the present embodiment, because of less heater elements 122, i.e. reduction in the number of parts, an advantageous effect of reducing manufacturing costs is obtained.

In the eleventh to the fifteenth embodiments described above, a through hole (121d) of an end cap (121) of a treatment portion (115) is formed so as to pass through the center of the end cap. As exemplified in the following embodiment, however, the through hole (121d) may be formed so as to pass through a portion other than the center of the end cap.

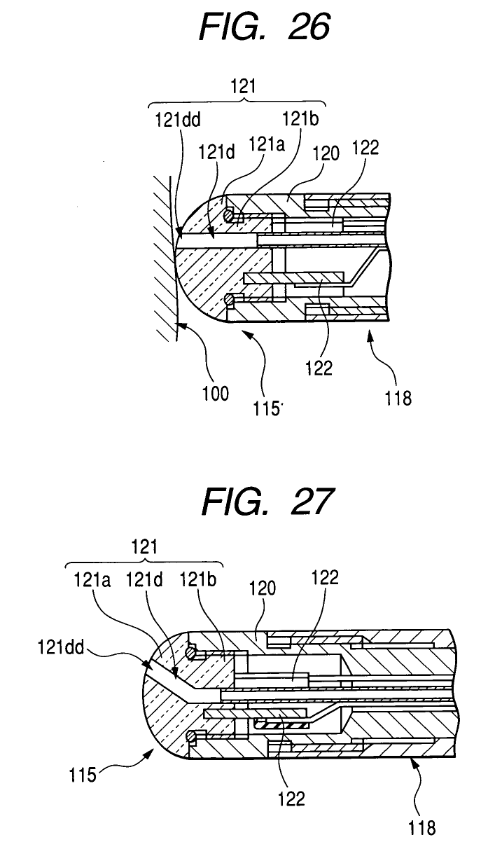

FIG. 25 is a vertical cross-sectional view of a portion (taken along the XVII-XVII line of FIG. 16) in the vicinity of the tip portion of a surgical treatment device according to a sixteenth embodiment of the present invention. FIG. 26 is a cross-sectional side view showing the inner configuration of the tip portion of the surgical treatment device according to the present embodiment.

The surgical treatment device according to the present embodiment includes substantially the same configuration as the surgical treatment device 111 of the eleventh embodiment. The only difference of the present embodiment from the eleventh embodiment is the position of the through hole 121d formed in the end cap 121 of the treatment portion 115. Thus, in the following description, the constructional elements of the present embodiment having the similar configurations to those of the constructional elements of the eleventh embodiment are referred to by the same reference numbers, and descriptions therefor are omitted.

As shown in FIGS. 25 and 26, the end cap 121 of the surgical treatment device according to the present embodiment includes three heater elements 122. As in the eleventh embodiment, these three heater elements 122 are disposed on the same circumference with an even interval therebetween.

The through hole 121d is formed at a position deviated, by a certain distance, from the central axis of the end cap 121 within a region surrounded by the three heater elements 122.

The configuration of the present embodiment is the same as the eleventh embodiment except as described above.

In the sixteenth embodiment as well, having the configuration as described above, the same effects as in the eleventh embodiment can be obtained. In addition, according to the present embodiment, an opening 121dd of the through hole 121d of the end cap 121 is disposed at a position deviated from the center of the end cap 121. As a result, during the treatment using the surgical treatment device, the opening 121dd is not blocked if the distal end of the end cap 121 comes into contact with the surface of tissues 100 as shown in FIG. 26. Thus, an advantage is provided such that air/liquid supply and suction operations can be carried out even in such circumstances.

In the eleventh to the sixteenth embodiments described above, a through hole may be provided at a position outside a region surrounded by the plurality of heater elements 122 which are disposed in the end cap 121, depending on the dimension of the outer diameter of the shaft 118 relative to the dimensions of internally arranged various constructional elements in the vicinity of a tip portion of the shaft 118.

Moreover, in the eleventh to the sixteenth embodiments, although a through hole (121d) is formed so as to extend parallel to the longitudinal axis of the shaft 118, the through hole (121d) may be formed so as to extend making a certain angle with the longitudinal axis of the shaft 118 as shown in the following seventeenth embodiment of the present invention.

FIG. 27 is a cross-sectional side view showing the internal configuration at a tip portion of a surgical treatment device according to the seventeenth embodiment of the present invention.

The surgical treatment device according to the present embodiment includes the same configuration as the surgical treatment device 111 of the eleventh embodiment. The only difference is the shape of the through hole 121d formed in the end cap 121 of the treatment portion 115. Thus, in the following description, the constructional elements of the present embodiment having the similar configurations to those of the constructional elements of the eleventh embodiment are referred to by the same reference numbers, and descriptions therefor are omitted.

As shown in FIG. 27, in the surgical treatment device according to the present invention, the through hole 121d is parallel to the longitudinal axis of the shaft 118 when resides in the protrusion 121b, however, when resides in the hemispherical portion 121a of the end cap 121, the through hole 121d is inclined with respect to the longitudinal axis of the shaft 118. As a result, a conduit which is gradually apart from the longitudinal axis of the shaft 118 is formed in the end cap 121.

The configuration of the present embodiment is the same as the eleventh embodiment except as described above.

According to the seventeenth embodiment having the configuration as described above, the same effects as in the sixteenth embodiment (wherein a through hole is formed at a position deviated from the center of an end cap) can be obtained.

In the eleventh to the seventeenth embodiments, the tip of an end cap (121) is formed to have substantially a hemispherical shape. The shape of a tip of an end cap is not limited to this, but may be varied as illustrated in the following eighteenth and nineteenth embodiments.

Surgical treatment devices according to the respective eighteenth and the nineteenth embodiments have substantially the same configuration as the surgical treatment device 111 according to the eleventh embodiment. The only difference of the eighteenth and the nineteenth embodiments from the eleventh embodiment is the shape of a tip portion of an end cap of a treatment portion. Thus, in the following description, the constructional elements of the eighteenth and the nineteenth embodiments having the similar configurations to those of the constructional elements of the eleventh embodiment are referred to by the same reference numbers, and descriptions therefor are omitted.

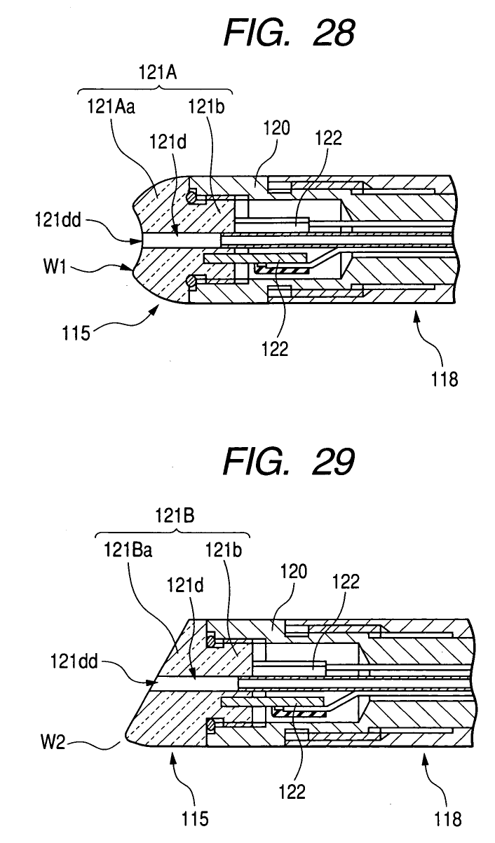

FIG. 28 is a cross-sectional side view showing the inner configuration of a tip portion of a surgical treatment device according to the eighteenth embodiment of the present invention.

A tip 121Aa of an end cap 121A according to the present embodiment has a concave shape rather than a hemispherical shape. The opening 121dd of the through hole 121d is positioned at approximately the center of the concave shape portion 121Aa.

The configuration of the present embodiment is the same as the eleventh embodiment except as described above.

In the eighteenth embodiment having the configuration as described above, blockage of the opening 121dd during treatment using the surgical treatment device can be prevented because a gap is produced between the surfaces of tissues and the opening 121dd if the tip portion (portion shown by W1 in FIG. 28) of the end cap 121 comes into contact with the surfaces of the tissues. Thus, an advantage is provided, as in the sixteenth and the seventeenth embodiments, that air/liquid supply and suction operations can also be carried out during treatment.

FIG. 29 is a cross-sectional side view showing the inner configuration of a tip portion of a surgical treatment device according to the nineteenth embodiment of the present invention.

An end cap 121B according to the present embodiment includes, at its tip, an inclined portion, i.e. a plane inclined with respect to the longitudinal axis of the shaft 118. The opening 121dd of the through hole 121d is formed approximately at the center of the inclined portion 121Ba.

The configuration of the present embodiment is the same as the eleventh embodiment except as described above.

In the nineteenth embodiment having the configuration as described above, the same effects as in the sixteenth to the eighteenth embodiments can be obtained.

Needless to say, the present invention is not limited to the embodiments described above, but may be embodied in various other modified forms without departing from the scope of the present invention.

1. A surgical treatment device comprising:
a shaft;
a treatment portion provided at a tip of the shaft for effecting heat treatment to living tissues;
a heater disposed within the treatment portion;
a cable member inserted through the shaft for supplying electricity to the heater; and
a heat insulating member inhibiting heat transfer from the treatment portion to the shaft.

2. The surgical treatment device according to claim 1, wherein the shaft has a mantle tube protecting the cable member, and the heat insulating member is disposed between the treatment portion and the mantle tube.

3. The surgical treatment device according to claim 1, wherein the heater includes a plurality of heater elements arranged on one circumference with a certain interval therebetween on a plane perpendicular to a longitudinal direction of the shaft.

4. The surgical treatment device according to claim 1, wherein the treatment portion has one of a hemisphere shape, a cylinder shape, a hook shape, and a needle shape.

5. A surgical treatment device comprising:
a shaft;
a treatment portion provided at a tip of the shaft for effecting heat treatment to living tissues;
a heater disposed within the treatment portion;
a cable member inserted through the shaft for supplying electricity to the heater; and
a heat insulating member inhibiting heat transfer from the treatment portion to the shaft,
a tip of the treatment portion having a conical shape.

6. A surgical treatment device comprising:
a shaft having a grip member at base end side thereof;
a treatment portion provided at a tip of the shaft for effecting heat treatment to living tissues, the treatment portion having a through hole opening to a front end thereof;
a heater disposed within the treatment portion;
a cable member inserted through the shaft for supplying electricity to the heater;
a first conduit member connected to the through hole;
a first connector provided at the grip member and connected to the cable member; and
a second connector provided at the grip member and connected to the first conduit member.

7. The surgical treatment device according to claim 6, wherein the heater includes a plurality of heater elements disposed in the treatment portion, and the through hole is formed in a portion of the treatment portion surrounded by the plurality of the heater elements.

8. The surgical treatment device according to claim 6, wherein an opening of the through hole and the first conduit member are located near an axis center of the shaft.

9. The surgical treatment device according to claim 6, wherein the shaft further includes a nozzle formed at a side thereof, a second conduit member connected to the nozzle, and a third connector provided at the grip member and connected to the second conduit member.

10. The surgical treatment device according to claim 6, further comprising a heat insulating member disposed between the treatment portion and the shaft for inhibiting heat transfer from the treatment portion to the shaft.

11. The surgical treatment device according to claim 6, wherein a tip of the treatment portion has one of a hemisphere shape, a concave shape, and a plane shape inclined with respect to a longitudinal axis of the shaft.

12. A surgical treatment system including a surgical treatment device and a liquid supply apparatus,
the surgical treatment device comprising:
a shaft having a grip member at a base end side thereof;
a treatment portion provided at a tip of the shaft for effecting heat treatment to living tissues, the treatment portion having a through hole opening to a front end thereof;
a heater disposed within the treatment portion; and
a cable member inserted through the shaft for supplying electricity to the heater;
a first conduit member connected to the through hole;
a first connector provided at the grip member and connected to the cable member; and
a second connector provided at the grip member and connected to the first conduit member,
the liquid supply apparatus comprising a liquid supply pump connected to the first conduit member through a liquid supply conduit connected to second connector.

13. A surgical treatment system including a surgical treatment device and a suction apparatus,
the surgical treatment device comprising:
a shaft having a grip member at a base end side thereof;
a treatment portion provided at a tip of the shaft for effecting heat treatment to living tissues, the treatment portion having a through hole opening to a front end thereof;
a heater disposed within the treatment portion; and
a cable member inserted through the shaft for supplying electricity to the heater;
a first conduit member connected to the through hole;
a first connector provided at the grip member and connected to the cable member; and
a second connector provided at the grip member and connected to the first conduit member,
the suction apparatus comprising a suction pump connected to the first conduit member through a suction conduit connected to second connector.

14. A surgical treatment system including a surgical treatment device and an air supply apparatus,
the surgical treatment device comprising:
a shaft having a grip member at a base end side thereof;
a treatment portion provided at a tip of the shaft for effecting heat treatment to living tissues, the treatment portion having a through hole opening to a front end thereof;
a heater disposed within the treatment portion; and
a cable member inserted through the shaft for supplying electricity to the heater;
a first conduit member connected to the through hole;
a first connector provided at the grip member and connected to the cable member; and
a second connector provided at the grip member and connected to the first conduit member,
the air supply apparatus comprising an air compressor connected to the first conduit member through an air supply conduit connected to second connector.

15. A surgical treatment system including a surgical treatment device, a liquid supply apparatus and a suction apparatus,
the surgical treatment device comprising:
a shaft having a grip member at a base end side thereof;
a treatment portion provided at a tip of the shaft for effecting heat treatment to living tissues, the treatment portion having a through hole opening to a front end thereof;
a heater disposed within the treatment portion;
a cable member inserted through the shaft for supplying electricity to the heater;
a first conduit member connected to the through hole;
a first connector provided at the grip member and connected to the cable member;
a second connector provided at the grip member and connected to the first conduit member;
a nozzle formed at a side of the shaft;
a second conduit member connected to the nozzle; and
a third connector provided at the grip member and connected to the second conduit member,
the liquid supply apparatus comprising a liquid supply pump connected to the second conduit member through a liquid supply conduit connected to the third connector,
the suction apparatus comprising a suction pump connected to the first conduit member through a suction conduit connected to second connector.

16. A surgical treatment system including a surgical treatment device, a liquid supply apparatus and an air supply apparatus,
the surgical treatment device comprising:
a shaft having a grip member at a base end side thereof;
a treatment portion provided at a tip of the shaft for effecting heat treatment to living tissues, the treatment portion having a through hole opening to a front end thereof;
a heater disposed within the treatment portion;
a cable member inserted through the shaft for supplying electricity to the heater;
a first conduit member connected to the through hole;
a first connector provided at the grip member and connected to the cable member;
a second connector provided at the grip member and connected to the first conduit member;
a nozzle formed at a side of the shaft;
a second conduit member connected to the nozzle; and
a third connector provided at the grip member and connected to the second conduit member,
the liquid supply apparatus comprising a liquid supply pump connected to the second conduit member through a liquid supply conduit connected to the third connector,
the air supply apparatus comprising an air compressor connected to the first conduit member through an air supply conduit connected to second connector.

17. A surgical treatment system including a surgical treatment device, and two of a liquid supply apparatus, an air supply apparatus and a suction apparatus,
the surgical treatment device comprising:
a shaft having a grip member at a base end side thereof;
a treatment portion provided at a tip of the shaft for effecting heat treatment to living tissues, the treatment portion having a through hole opening to a front end thereof;
a heater disposed within the treatment portion; and
a cable member inserted through the shaft for supplying electricity to the heater;
a first conduit member connected to the through hole;
a first connector provided at the grip member and connected to the cable member;
a second connector provided at the grip member and connected to the first conduit member;
a nozzle formed at a side of the shaft;
a second conduit member connected to the nozzle; and
a third connector provided at the grip member and connected to the second conduit member,
the second connector being connected to one of a liquid supply pump of the liquid supply apparatus, an air compressor of the air supply apparatus and a suction pump of the suction apparatus through an conduit,
the third connector being connected to the other one of the liquid supply pump of the liquid supply apparatus, the air compressor of the air supply apparatus and the suction pump of the suction apparatus through an conduit.
